(19) United States
(12) Patent Application Publication
(10) Pub. No.: US 2011/0014226 A1
(43) Pub. Date: Jan. 20, 2011

(54) HIGH THROUGHPUT PROTEIN INTERACTION ASSAY

(76) Inventors: Michael J. Caulfield, West Point, PA (US); Michael D. Miller, Chalfont, PA (US); Joseph G. Joyce, Lansdale, PA (US); Paul D. Zuck, Trenton, NJ (US); Krista L. Getty, Quakertown, PA (US); Vadim Dudkin, Lansdale, PA (US); Elizabeth A. Ottinger, Harleysville, PA (US)

Correspondence Address: MERCK, P O BOX 2000, RAHWAY, NJ, 07065-0907, US

(21) Appl. No.: 12/921,890

(22) PCT Filed: Mar. 17, 2009
(86) PCT No.: PCT/US09/37390

§ 371 Date: Sep. 10, 2010

Related U.S. Application Data
(60) Provisional application No. 61/069,803, filed on Mar. 18, 2008.

(51) Int. Cl. A61K 39/385; C40B 30/04; A61K 39/21; A61K 39/29; A61K 39/095; A61K 39/00; C07D 401/12; C07D 211/60; A61P 37/04; A61P 31/18; A61P 31/14; A61P 31/04; A61P 37/02; A61P 29/00; A61P 3/10; A61P 25/00

(52) U.S. Cl. 424/196.11; 506/9; 424/208.1; 424/228.1; 424/250.1; 424/197.11; 424/184.1; 544/365; 546/189; 546/208; 546/225

(57) ABSTRACT

A method to identify small molecules useful as therapeutics and/or vaccines to prevent, alleviate or ameliorate a pathogenic infection or an autoimmune disorder. The method can be used to screen small molecule test compounds for the ability to disrupt particular antigen-antibody interactions of interest. In one embodiment, the antigen is a pathogen-derived antigen and the antibody decreases or inhibits virulence of the pathogen when bound to the antigen (e.g., a neutralizing antibody, antibody with serum bactericidal activity, etc.). In another embodiment, the antigen is a self-antigen (autoantigen) and the antibody is an autoantibody that is known to be associated with a pathological condition (e.g., autoimmune disorder). Compounds that bind to the antigen or antibody disrupt binding can be used as therapeutics to decrease or inhibit the autoimmune disorder.

### 1. FIELD OF INVENTION

The present invention relates to a platform to rapidly screen large collections of small molecules for identification of compounds for use as treatments and/or vaccines for infectious diseases and treatments for autoimmune disorders. The invention also encompasses small molecule compounds identified by the assay.

### 2. BACKGROUND

### 2.1 Vaccines

One well-established vaccinology strategy for human pathogens is the use of subunit vaccines in which a biomolecular component of the pathogen (such as a protein, lipoprotein or polysaccharide) is presented to the host immune system in a form which can elicit a robust primary and memory recall antibody response. A variation of this approach is to use defined segments of a pathogen subunit when it is known that such segments are recognized by neutralizing antibodies produced during natural infection and capable of killing or decreasing the virulence of the pathogen (Haro and Gomara, 2004, Curr Protein Pept Sci. 5:425-33 and Wang, 2006, Curr Opin Drug Discov Devel. 9:194-206).

The success of a sub-unit approach for certain bacterial and viral pathogens has remained frustratingly elusive due in large part to evolutionary-adapted immune-evading defense mechanisms of the pathogens (Pitisuttithum et al., 2006, J Infect Dis. 194:1661-71 and Yang et al., 2005, PNAS 102:797-801). One such mechanism is the presentation of immuno-dominant surface proteins containing hypervariable recognition sequences. Immunization with these sub-unit proteins elicits a robust antibody response, but this response is often limited to the strain-specific protein sequence. Accordingly, cross-protection against distant or even closely-related strains is poor. Often, highly conserved sequences which mediate important functional roles in the pathogen life cycle are well-shielded from immune recognition either by structural inaccessibility or epitope masking via glycosylation (Vigerust and Shepherd, 2007, Trends Microbiol. 15:211-8 and Srivastava et al., 2005, Hum Vaccin. 1:45-60). In cases where conserved epitopes may be permanently or even transiently accessible, a further complicating factor can involve proper structural presentation. In the case of structurally-sensitive conformational epitopes, it is often not possible or very difficult to reproduce these epitopes in the context of synthetic peptides or even purified full-length protein.

Past biomolecule mimotope approaches have often focused on discovery of small peptides as mimotopes of polysaccharide, carbohydrate, protein, or toxin structures (Kieber-Emmons et al., 1997, Curr Opin Biotechnol. 8:435-41 and Harvey et al., 2005, Bioorg Med Chem Lett. 15:3193-6). Typically, screening approaches utilizing phage-display libraries have been employed for identification of peptide leads, with further optimization effected through synthetic manipulations of the sequence. Some instances of synthetic combinatorial libraries for di- and isopeptide mimetics have also been described (Falciani et al., 2005, Chem Biol. 12:417-26; Pinilla et al., 1999, Curr Opin Immunol. 11:193-202). While these approaches have generated some measure of success, the library construction and panning steps can be labor-intensive and inefficient. Furthermore, whereas peptide mimotopes of proteins or polysaccharides can be identified using this approach, these peptides do not represent highly constrained molecular species.

### 2.2 Autoimmune Disorders

Most recognized autoimmune disorders (Ads) cannot yet be treated directly. Therapy often takes the form of supportive care for disease symptoms (e.g., use of corticosteroids or NSAIDS for inflammatory responses, etc.) or the use of generalized immunosuppressive agents such as cyclophosphamide (Richman and Agius, 2003, Neurology 61:1652-61). These therapies are non-specific and are associated with a significant degree of adverse or off-target effects, particularly with regard to immunosuppressive approaches. A variety of biological immunotherapy approaches utilizing monoclonal antibodies which target specific cellular populations or cytokines have also been investigated (Hasler, 2006, Springer Semin Immunopathol. 27:443-56 and Prete et al., 2005, Clin Exp Med. 5:141-60). More recently, active immunotherapy approaches have been introduced in which vaccination strategies using peptide mimetics of surface ligands or anti-idiotypic antibody administration (Wraith, 2006, Eur J Immunol. 36:2844-8; McDevitt, 2004, PNAS 101, Suppl 2:14627-30).

The design of low molecular weight ligands that disrupt protein-protein interactions has remained a challenging endeavor (see, e.g., Cochran, 2000, Chem. Biol. 7:R85-R94), Conventional means of identifying small molecules from chemical libraries that are inhibitors of protein-protein interactions have resulted in limited success (Degterev et al., 2001, Nat. Cell. Biol. 3:173-182; Debnath et al., 1999, J. Med. Chem. 42:3203-3209; Qureshi et al., 1999, PNAS 96:12 156-12 161; Tian et al., 1998, Science 281:257-259; Tilley et al., 1997, J. Am. Chem. Soc. 119:7589-7590).

### 3. SUMMARY

The present invention relates to a method to identify small molecules that inhibit particular antibody-antigen interactions of interest. In one embodiment, the antigen is a pathogen-derived antigen and the antibody decreases or inhibits virulence of the pathogen when bound to the antigen (e.g., a neutralizing antibody, antibody with serum bactericidal activity, etc.). In another embodiment, the antigen is a self-antigen (autoantigen) and the antibody is an autoantibody that is known to be associated with a pathological condition (e.g., autoimmune disorder). Test compounds are incubated with the antigen and antibody in order to identify those that can decrease or inhibit binding. Compounds that bind to the antigen to disrupt antibody binding are termed “Class 1 compounds” while compounds that bind to the antibody and disrupt its ability to bind antigen are termed “Class 2 compounds”.

Compounds identified by the methods of the invention can be used, e.g., as therapeutics, vaccines, research tools (e.g., to study binding characteristics of the antibody or antigen to which it binds or to identify mimetics of the antigen or antibody to which it binds), and/or diagnostics (e.g., to detect the presence and/or quantity of the antibody or antigen to which it binds). In embodiments where the antigen is pathogen-derived, Class 1 compounds can be administered to a patient in need thereof as an anti-infective therapeutic. Class 2 compounds can be administered as a prophylactic or therapeutic vaccine against the pathogen. In embodiments where the antigen is a self-antigen, Class 1 and Class 2 compounds can be administered to a patient in need thereof as antibody antagonists to disrupt autoantibody binding to alleviate or ameliorate the pathological condition.

### 4. BRIEF DESCRIPTION OF THE FIGURES

FIG. 1 schematically depicts the D5 Competitive Binding Assay (DCBA). A biotinylated derivative of recombinant 5-helix peptide (a gp41 conformational mimetic molecule that presents the hydrophobic pocket in a stabilized structural context) is incubated with monoclonal antibody D5 conjugated to europium cryptate (Eu-D5) and streptavidin-conjugated allophycocyanin. The biotin-5-helix binds to the streptavidin-conjugated allophycocyanin to form a complex. When Eu-D5 binds to the hydrophobic pocket of the biotin-5-helix complex, it brings the Eu into close proximity with the APC. Excitation of Eu at 340 nm results in emissions at 620 nm which in turn causes excitation of APC that results in long-lived emissions at 665 nm through fluorescence resonance energy transfer (FRET) from Eu to APC. Addition of a compound that disrupts 5-helix-D5 binding will not change emissions at 620 nm but will decrease emissions at 665 nm because Eu is no longer in close enough proximity to APC to produce FRET, and as a result, the ratio of fluorescence counts measured at 665 nm wavelength versus those at 620 nm wavelength will decrease.

Abbreviations: Eu=europium cryptate; Bio=biotin; SA=streptavidin; APC=allophycocyanin

FIG. 2 depicts the three classes of compounds which can inhibit binding of D5 to 5-helix in the DCBA assay. Class 1 are molecules which bind to the hydrophobic pocket on 5-helix (labeled A). Class 2 are molecules which bind to the CDR region of D5 (labeled B). Class 3 are molecules which belong to neither Class 1 nor Class 2 that can interfere with the assay readout through interaction with any component of the biotin-streptavidin-allophycocyanin complex or with europium cryptate (labeled C).

FIG. 3 depicts a counter-screen to identify Class 3 compounds. The DCBA assay is repeated using monoclonal antibody F19 instead of D5. F19 is an antibody which binds to one of the scaffolding outer helical bundles on 5-helix, but in close enough proximity to effect an energy transfer event to APC when labeled with Eu.

FIG. 4 depicts a molecular model of boc-Aha derivatized Compound 2.4 fitting into the D5 antibody combining site. Molecular modeling was performed utilizing the published crystal structure of D5 bound to 5-helix. In this pose, Compound 2.4 is shown overlaid on the three 5-helix residues that make critical contacts in the CDR pocket of the antibody. Groups on the hapten which mimic amino acids on 5-helix which make critical contacts within the D5 CDR are circled (circle 1 shows that naphthyl mimics Trp 571 on 5-helix; circle 2 shows that benzyl mimics Leu 568 on 5-helix; circle 3 shows that urea mimics Gln 575 on 5-helix).

FIGS. 5A-5B depict antibody responses to DCBA hapten-CRM₁₉₇ protein conjugates in mice. Shown are serum titrations against 5H from individual mice from the experiment described in Table 1 in which mice were immunized 3 times with (A) Compound 2.45 conjugated to CRM₁₉₇ or (B) Compound 2.58 conjugated to CRM₁₉₇.

### 5. DETAILED DESCRIPTION

The present invention relates to a platform to rapidly screen large collections of small molecules for identification of compounds that inhibit particular antibody-antigen interactions of interest. The small molecules identified as inhibitors have different uses including use as a research tool to study antibody antigen interaction and use as a reagent to detect the presence of either antibody or antigen. Small molecule can also potentially be used as treatments and/or vaccines for infectious diseases and treatments for autoimmune disorders.

As used herein, “small molecule” refers to an organic molecule of different sizes. In one embodiment, the small molecule is an organic molecule that is not a polypeptide, nucleic acid, or lipid.

### 5.1 The Assay

The assay of the present invention comprises an antigen component and an antibody component that binds to the antigen component. The antigen component can be any type of biomolecule including, but not limited to, polypeptides, peptides, polysaccharides, and carbohydrates. The antigen component can be a full length biomolecule or fragment or conformational mimetic thereof. Fragments and conformational mimetics provide an epitope of the antigen biomolecule bound by the antibody. In one embodiment, the antigen is a pathogen-derived antigen and the antibody decreases or inhibits virulence of the pathogen when bound to the antigen (e.g., a neutralizing antibody, antibody with serum bactericidal activity, etc.) (see Section 5.2). In another embodiment, the antigen is a self-antigen (autoantigen) and the antibody is an autoantibody that is known to be associated with a pathological condition (e.g., autoimmune disorder) (see Section 5.3). The antibody component can be of any immunoglobulin class and/or isotype (i.e., IgG1, IgG2, IgG3, IgG4, IgM, IgA, IgD, IgE and IgM) and can be a full length molecule or a biologically relevant fragment or specific binding member thereof, including but not limited to, Fab, F(ab′)2, Fv and scFv, which is capable of binding substantially the same epitope as in the full length biomolecule.

Binding of the antibody to the antigen provides a “binding signal” that can be detected by any method known in the art. In preferred embodiments, at least one of the components of the assay is modified to incorporate a molecular moiety capable of detection (including, but not limited to, radioactive isotopes, enzymes, luminescent agents, fluorescent agents, and dyes) to aid in the detection of binding. In a preferred embodiment, the binding is detected using a fluorescence resonance energy transfer (FRET) format.

Test compounds are incubated with the antibody and antigen of the assay under conditions which allow for antigen-antibody binding. As used herein, the term “test compound” refers to a small molecule that is tested in the assay for the ability to disrupt antigen-antibody binding.

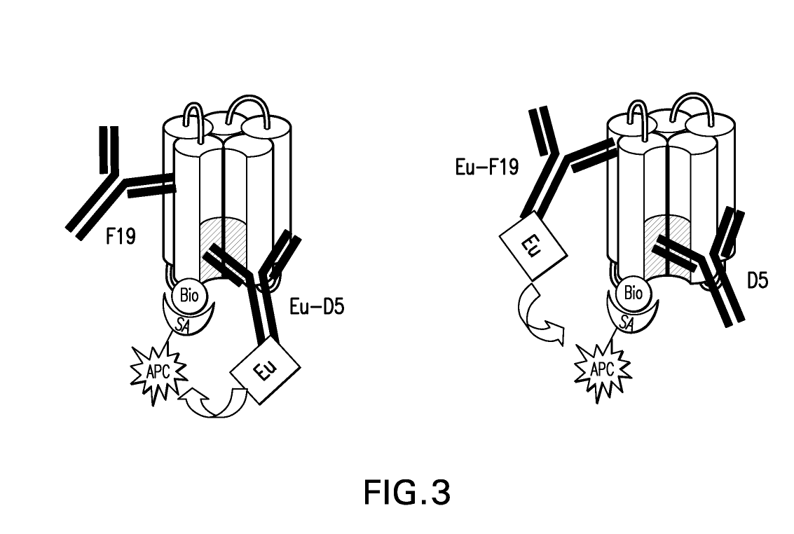

A test compound that decreases the binding signal can do so by disrupting the antibody-antigen interaction of the antigen and antibody to be a specific antagonist or by interfering with the generation or detection of the binding signal to be a non-specific antagonist. Non-specific antagonists are not of interest in the present invention. Accordingly, test compounds that have been shown to decrease binding signal in the primary assay can be assayed for the specificity of their antagonism. In embodiments using FRET to generate the binding signal, a counter-screening antibody labeled with the same fluorescent agent as the antibody component of the assay can be used. A counter-screening antibody is an antibody that binds to the antigen at a site that is distinct from the site bound to by the antibody component of the assay but is close enough to the fluorescent agent on the antigen to effect energy transfer (see, e.g., FIG. 3). A test compound that can decrease binding signal of an antigen-counter-screening antibody complex is a non-specific antagonist.

Those test compounds that decrease the binding signal and are specific antagonists are candidate compounds. As used herein, the term “candidate compound” refers to a test compound that does specifically bind to a component in the assay to disrupt binding. Test compounds that bind to a component of the assay in such a manner as to alter the binding signal by a means other than disruption of antigen-antibody binding (e.g., by binding to the signal producing molecule, or by causing an allosteric affect, etc.) are not candidate compounds. Candidate compounds are further tested in a secondary screen to determine which component of the assay is bound (i.e., the antigen or antibody). Any method known in the art can be used to determine binding in the secondary screen including, but not limited to, surface plasmon resonance (e.g., a biacore assay), ELISA, and functional assays.

Compounds that bind to the antigen to disrupt antibody binding are termed “Class 1 compounds” while compounds that bind to the antibody and disrupt its ability to bind antigen are termed “Class 2 compounds”. Compounds that alter the binding signal by a means other than disruption of antigen-antibody binding (i.e., non-specific antagonists) are termed “Class 3 compounds”. Class 3 compounds can interfere with the assay readout, e.g., through interaction with any component of the biotin-streptavidin-allophycocyanin complex or with europium cryptate and thus inhibit effective energy transfer from the Eu label.

In some embodiments, the assay conditions are altered to favor the identification of either Class 1 or Class 2 compounds.

### 5.2 Anti-Infectives

In one embodiment, the assay of the invention is used to identify small molecule compounds useful as anti-infectives or research tools. The antigen component of the assay is a pathogen-derived antigen (including fragments) or conformational mimetic thereof. Any pathogen can provide the antigen including, but not limited to, HIV, HCV, and Neisseria meningitides, or biological subunit thereof. The antibody component of the assay decreases or inhibits virulence of the pathogen when bound to the antigen (e.g., a neutralizing antibody, antibody with serum bactericidal activity, etc.) using an in vivo or in vitro assay. As used herein, the term “neutralizing antibody” refers to an antibody with the ability to reduce the likelihood or severity of infection by at least one strain or isolate of a pathogen (e.g., virus, bacteria, etc.) in a cell culture or patient. In one embodiment, an antibody is determined to neutralize a specific strain or isolate of a pathogen if the IC₅₀ for that antibody is in the range of up to about 100 μM. In preferred embodiments, the IC₅₀ for a neutralizing antibody is less than 50 μM and preferably less than 10 μM.

In a preferred embodiment, the antigen component of the assay is HIV gp41, a fragment, or conformational mimetic thereof. The antibody component of the assay in this embodiment binds to the hydrophobic pocket of gp41. In a more preferred embodiment, the antigen component is 5-helix and the antibody component is D5 (see, e.g., Root et al., 2001, Science 291:884-888; Root and Hammer, 2003, PNAS 100:5016-5021; Miller et al., 2005, PNAS 102:14759-14764; Steger and Root, 2006, J Biol Chem 281:25813-25821; and International Publication No. WO2005/118887 for more detailed description of assay components).

Once the HIV cell receptor gp120 interacts with cellular CD4 and a chemokine co-receptor, gp41 is exposed. Conformational changes in gp41 ensue and allow HIV to fuse with a target cell membrane and enter the cell (for a review, see Eckert and Kim, 2001, Annual Review Biochemistry 70: 777-810). During this gp41-mediated membrane fusion process, the ecto-domain of gp41 transitions through various conformational intermediates that are believed to include a pre-hairpin structure. This pre-hairpin intermediate exposes the N-terminal fusion peptide which inserts into the target cell membrane. The ecto-domain proceeds to form a hairpin structure, which results in the juxtaposition of the target cell plasma membrane and the virion envelope. Gp41 exists in a trimeric state on the surface of the virion. The gp41 ecto-domain contains two distinct heptad repeat (HR) regions, designated HR1 and HR2. The HR1 region from three independent gp41 proteins interact with each other to form a trimeric coiled-coil structure that exposes on its surface three symmetrical grooves. To form the trimeric hairpin structure (or six-helical bundle), the HR2 regions fold back and interact with the grooves present on the surface of the coiled-coil structure. In the groove, the C-terminal halves of adjacent HR1 segments form a hydrophobic pocket which accommodates three key residues from the N-terminal portion of the HR2 region. The integrity of this pocket is critical for fusion and HIV infectivity.

5-helix is a conformational mimetic of gp41 that presents the gp41 hydrophobic pocket in a stabilized structural context. 5-Helix is a recombinantly-produced construct composed of a series of three alternating HR1 peptides and two HR2 peptides derived united by small peptidic linkers. This recombinant peptide spontaneously folds into a 5-helical bundle in which one of three potential grooves formed by the HR1 trimer is presented in an exposed and highly stabilized context. The amino acid sequence of the peptides that make up 5-helix is disclosed in International Publication No. WO2005/118887.

The epitope for D5 antibody binding lies in the hydrophobic pocket region located near the carboxy terminal half of the HR1 trimer. Amino acids L568, W571 and K574 of gp41 are critical for antibody binding while V570 contributes to a lesser extent. By interacting with the hydrophobic pocket of the HR1 region, D5 IgG possesses the functional capacity of preventing the in vitro interaction of the N- and C-peptides. Antibody D5 inhibits HIV fusion with target cell membranes by interfering with the intramolecular interactions occurring between the gp41 HR1 and HR2 regions that lead to the formation of the 6-helical bundle. The amino acid sequence of the antigen binding portions of D5 is disclosed in International Publication No. WO2005/118887.

When 5-helix is used in conjunction with D5, the assay is termed “D5 competitive binding assay” or “DCBA”.

In another preferred embodiment, the antigen component of the assay is the Neisseria meningitides capsular polysaccharide, a fragment, or conformational mimetic thereof. The antibody component of the assay in this embodiment binds to the polysialic acid component of the capsule and, preferably, has little or no reactivity to NCAM-associated polysialic acid.

In a specific embodiment, conditions favoring hydrophobic binding can be used to favor the identification of Class 1 compounds in the DCBA assay. In another specific embodiment, conditions less favorable for hydrophobic binding can be used to favor the identification of Class 2 compounds in the DCBA assay.

### 5.2.1 Class 1 Compounds

Small molecules that decrease or inhibit the binding signal in the assay by binding to the antigen component and interfering with antibody binding are Class 1 compounds. Such compounds are useful as potential therapeutics because they bind to the antigen component in the same place as an antibody that has the ability to decrease or inhibit virulence of the pathogen when bound to the antigen.

Any method known in the art can be used to determine if a compound is a Class 1 compound including, but not limited to, surface plasmon resonance (e.g., a biacore assay), ELISA, and functional assays (e.g., The Viral Entry, Reverse Transcription, and Integration: Cellular Assay for Leads or VERTICAL, see infra).

In embodiments where the assay is a DCBA assay, a VERTICAL assay can be conducted as a secondary screen to identify positive DCBA lead compounds which bind to the hydrophobic pocket region of 5-helix and act to inhibit viral entry. Briefly, HeLa cells expressing cell surface receptors required for HIV attachment and entry also contain an integrated β-galactosidase reporter gene under control of an HIV LTR promoter. The cells are incubated with HIV in the presence and absence of the test compounds. After 48 h of infection, cells are lysed, and the β-galactosidase activity (which is indicative of the viral replication level) is detected (see, e.g., Section 6.5). Compounds that were positive in the primary DCBA screen but do not decrease β-galactosidase activity in the VERTICAL screen may be Class 2 compounds (bind D5) rather than Class 1 compounds (bind 5-helix).

In order to determine whether the test compounds that decrease β-galactosidase activity specifically block the entry step, the VERTICAL assay is conducted using HIV viruses that either use a different mechanism to enter the cell (e.g., an HIV-1 virus pseudo-typed with the VSV-G envelope protein that uses the VSV-G entry route) or that has a mutation in the gp41 hydrophobic pocket such that test compound binding is compromised (e.g., point mutations at L568A, V570A, and/or K574A in gp41).

5.2.2 Class 2 Compounds

Small molecules that decrease or inhibit the binding signal in the assay by binding to the antibody component and interfering with antigen binding are Class 2 compounds. Such compounds are useful as immunogens for prophylactic or therapeutic vaccines or research tools. The Class 2 compounds bind to an antibody known to decrease or inhibit virulence of a pathogen when bound to the pathogen and thereby mimic the structure of the neutralizing epitope. Such compounds are termed “mimotopes”. As used herein, the term “mimotope” refers to a small molecule that mimics the epitope to which an antibody binds. The mimotope binds to the complementarity determining region (CDR) of the antibody. As such, a mimotope is a competitive inhibitor with the endogenous antigen for antibody binding. When a mimotope is administered to an individual under conditions that elicit an immune response, antibodies can be endogenously produced that bind to the same or substantially the same epitope as the antibody used to originally isolate the mimotope. When a mimotope is administered to an individual under conditions that do not elicit an immune response, the mimotope can act as an antagonist and disrupt antigen-antibody binding.

Because of their size, small molecule mimotopes display a much more limited amount of molecular flexibility when compared to an extended polypeptide sequence (Dias et al., 2006, J Am Chem Soc. 128:2726-32; Freeman et al., 2005, J Biol Chem. 280:8842-9; Kelso et al., 2004, J Am Chem Soc. 126:4828-42; O'Leary and Hughes, 2003, J Biol Chem. 278:25738-44). Furthermore, many small molecules generated from large combinatorial libraries contain a variety of functionalized heterocyclic ring systems, further restricting the conformational space available to them. For small molecules acting as mimotopes of biomolecules this restricted structural flexibility offers an advantage from an immunological viewpoint in terms of conformational display since the majority of the response would be directed toward the appropriate and desired form of the epitope. Additionally, the small size of the mimotope reduces the misdirection of the immune response to non-neutralizing epitopes.

For gp41, several highly structured and stabilized peptide-based mimics of the helical trimer-of-hairpins core have been described, including 5 and 6-helix bundles (Root et al., 2001, Science 291:884-888; Steger and Root, 2006, J Biol Chem 281:25813-25821; Root and Hammer, 2003, PNAS 100:5016-5021) and N-peptide trimers (Luftig et al., 2006, Nat Struct. Mol Biol. 13:740-7; Bianchi et al., 2005, PNAS 102:12903-8; Eckert and Kim, 2001, 98:11187-92). However, as potential vaccine candidates these molecules share similar drawbacks to those outlined supra as they are still prone to induction of immune responses to regions not critical for neutralization and conformational flexibility of critical neutralization epitopes.

Any method known in the art can be used to determine if a compound is a Class 2 compound including, but not limited to, surface plasmon resonance (e.g., a biacore assay), ELISA, and functional assays (e.g., The Viral Entry, Reverse Transcription, and Integration: Cellular Assay for Leads or VERTICAL).

Structure A (see Section 5.2.3) is a genericized structure representing a subset of Class 2 compounds identified by the DCBA screen. Compounds 1.1-1.12 (see Table 2) are examples of Class 2 compounds that are encompassed by Structure A.

Class 2 compounds identified by the methods of the invention to be used as immunogens are preferably coupled to a carrier protein. The identified compounds are derivatized in order to accommodate coupling to the carrier protein. The conjugation method should be selected based on the characteristics of the carrier protein and the compound to be conjugated. The choice of chemistries available for conjugation of the Class 2 compounds to carrier proteins is broader than that available for biomolecules (such as polysaccharides and peptides) as it is not limited by the available functionalities inherent in amino acid and carbohydrate structures. Additionally, the linkage should not interfere with the ability of the compound to bind to the antibody that was used in the screen in which the compound was isolated (isolating antibody). The Class 2 compound can be co-crystallized with the isolating antibody or modeled in silico using existing crystal structures of the isolating antibody bound to its epitope. This data can aid in selection of the best sites for compound derivatization and conjugation. Alternatively, sites for derivatization and conjugation can be empirically determined.

Any method of coupling can be used to link the compound and carrier protein providing that it is compatible with the functional groups targeted for coupling. Non-limiting examples of coupling methods include, but are not limited to, maleimide/thiol coupling, bromoacetamide/thiol coupling, reductive amination, and various “click” chemistries (see Kolb et al., 2001, Angew Chem Int Ed 40:2004-21 and Kolb and Sharpless, 2003, Drug Discovery Today 24:1128-37) such as azide-alkyne coupling, aminooxy/oxime coupling, and enzyme-mediated coupling (see Tanaka et al., 2005, FEBS Lett 579:2092-6). In a preferred embodiment, maleimide/thiol coupling is used. In a more preferred embodiment, a 6-aminohexanoic acid linker arm is added to the compound of interest. The linker system can contain a thiol or thiol-reactive functional group to allow for coupling to either a maleimidated or thiolated carrier protein, respectively.

Any carrier protein known in the art can be used. In preferred embodiments, the carrier protein can be selected from the group consisting of OMPC (Outer Membrane Protein Complex of Neisseria meningitides; see e.g., U.S. Pat. Nos. 4,271,147; 4,707,543; and 5,494,808, each of which is incorporated by reference in its entirety), BSA (bovine serum albumin), OVA (ovalbumin), THY (bovine thyroglobulin), KLH (keyhole limpet hemocyanin), tetanus toxoid, HbSAg (surface antigen protein) and HBcAg (core antigen protein) of Hepatitis B virus, rotavirus capsid proteins, L1 protein of the human papilloma virus, diptheria toxoid, C. diphtheriae CRM197 protein, flagellin, and human papillomavirus VLP (virus-like particle) type 6, 11 and 16. In an embodiment, OMPC is the carrier protein.

Preferably, carrier protein conjugated mimotopes are administered with an adjuvant (see Section 5.5).

5.2.3 Compounds that Bind Antibody D5 in the DCBA Assay

Structure A is a genericized structure representing a subset of the Class 2 compounds identified by the DCBA screen as binding the D5 antigen binding region. Compounds 1.1-1.12 (see Table 2) and Compounds 2.1-2.61 (Table 3) are examples of Class 2 compounds that are encompassed by Structure A.

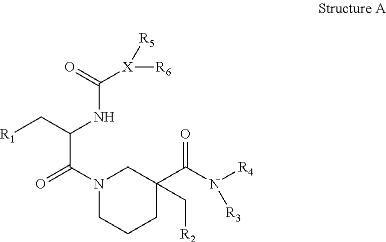

R₁ is an optionally substituted aryl or heteroaryl;
R₂ is an optionally substituted aryl or heteroaryl;
R₃ is either H or a C₁-C₆ alkyl;
R₄ is either H or a C₁-C₆ alkyl;
X is either N or C;
R₅ is selected from the group consisting of:

### H,

(C═O)OC₁-C₆ alkyl,
(C═O)OC₁-C₆ cyloalkyl,
(C═O)OC₁-C₆ aryl,
(C═O)OC₁-C₆ heterocyclyl,
(C═O)OC₁-C₆ alkyl,
aryl,
C₂-C₆ alkenyl,
C₂-C₆ alkynyl,
heterocyclyl,
C₃-C₆ cycloalkyl,

### SO₂R^{a}, and

### (C═O)NR^{b}₂,

said alkyl, cycloalkyl, aryl, heterocylyl, alkenyl, and alkynyl is optionally substituted with one or more substituents selected from R₇,
R₆ is selected from the group consisting of

### H,

C₍₀₋₆₎alkyl optionally substituted with a heterocyclic ring or aryl,
said alkyl, heterocyclic ring and aryl is optionally substituted with R^{a}; or
R₅ and R₆ together with X form a monocyclic or bicyclic ring with 5-7 members in each ring and,
when X is C, it optionally contains a 1-4 heteroatoms selected from N, O and S, and
when X is N, it optionally contains 1 to 4 additional heteroatoms selected from N, O and S, said monocylcic or bicyclic ring optionally substituted with one or more substituents selected from R₇;
R₇ is selected from the group consisting of:
(C₁-C₆)alkyl,
(C═O)ᵣOₛ (C₁-C₆)alkyl,
Oᵣ(C₁-C₃)perfluoroalkyl,

### S(O)ₘR^{a},

### SR^{a},

(C₁-C₆)alkylene-S(O)ₘR^{a},
oxo,

### OH,

halo,

### CN,

(C₂-C₆)alkenyl,
(C₂-C₆) alkynyl,
(C₃-C₆)cycloalkyl,
aryl
(C₁-C₆)alkylene-aryl,
heterocyclyl,
(C₁-C₆)alkylene-heterocyclyl

### N(R^{b})₂,

(C₁-C₆)alkylene-N(R^{b})₂,

### C(O) R^{a},

### CO₂ R^{a},

(C₁-C₆)alkylene-CO₂ R^{a},

### C(O)H, and

### CO₂H

(C₁-C₆)alkylene-CO₂H
said alkyl, alkenyl, alkynyl, alkylene, cycloalkyl, aryl, and heterocyclyl is optionally substituted with up to three substituents selected from R^{b}, OH, (C₁-C₆)alkoxy, halogen, CO₂H, CN, O(C═O) (C₁-C₆) alkyl, oxo, and N(R^{b})₂; and wherein each R is independently 0 or 1, each s is independently 0 or 1, and each m is independently 0, 1, or 2
R^{a} is selected from the group consisting of:
(C₁-C₆) alkyl,
(C₃-C₆) cycloalkyl,
aryl,
or heterocyclyl; and
R^{b} is selected from the group consisting of:

### H,

(C₁-C₆) alkyl,
aryl,
heterocyclyl,
(C₃-C₆) cycloalkyl,
(C═O)O(C₁-C₆)alkyl,
(C═O)(C₁-C₆)alkyl or

### S(O)₂ R^{a}.

If indicated, the aryl can be optionally substituted. Examples of aryls include phenyl, naphthyl, tetrahydronaphthyl (tetralinyl), indenyl, anthracenyl, and fluorenyl, each of which can be optionally substituted.

The term “aryl” refers to an aromatic group selected from the group consisting of 5-, 6- or 7-membered aromatic rings, 8-, 9- or 10-membered bicyclic aromatic rings, and 11- to 15-membered tricyclic rings aromatic rings.

The term “heteroaryl” refers to a 5- or 6-membered heteroaromatic ring containing from 1 to 4 heteroatoms independently selected from N, O and S, wherein each N is optionally in the form of an oxide and each S in a ring which is not aromatic is optionally S(O) or S(O)₂.

Reference to C₀-C₆ can mean that there is no intervening alkyl between two molecules (as denoted by C₀) or that the alkyl is a univalent radical containing 1 to 6 carbon atoms saturated with hydrogen atoms (as denoted by C₁-C₆), which can be optionally substituted. The C₁-C₆ alkyl can be arraigned in a linear or branched chain. Examples include methyl, ethyl, propyl, butyl, t-butyl. Substituted C₁-C₆ alkyl can be halo-C₁-C₆ alky (e.g., containing 1-6 halogens, each of with is preferably Fl or Cl), and C₁-C₃-heterocyclic group (e.g., ethyl-piperidine), the alkyl portion can be in a linear or branched chain and depending on the indicated number of carbons may include methyl, ethyl, propyl, butyl, t-butyl.

A “heterocyclic ring” is a 5- to 7-membered saturated or unsaturated non-aromatic carbocyclic ring having 1, 2, 3 or 4 heteroatoms as part of the ring. If indicated, the heterocyclic ring can be optionally substituted. Each heteroatom is independently N, O or S, and is attached through a ring carbon or nitrogen. Examples of heterocyclic rings include piperidine which may be optionally substituted.

Reference to “optionally substituted” to aryl, heteroaryl, heterocyclic groups, or specific types of aryl, heteroaryl, or heterocyclic ring, indicates either zero substituents or one or more substituents. Each substituent is independently selected from the group consisting of halogen atoms, —OR⁸, —SR⁸, —N(R⁸)₂, —N(C₁-C₆ alkyl)O(C₁-C₆ alkyl), C₁-C₆ alkyl, C₁-C₆ haloalkyl, halo(C₁-C₆ alkoxy), —NO₂, —CN, —CF₃, —SO₂(C₁-C₆ alkyl), —S(O)(C₁-C₆ alkyl), —CO₂R⁸, —C(O)R⁸, and —CON(R⁸)₂, and 2 adjacent substituents of said aryl and/or heteroaryl groups are optionally taken together to form a 3- to 6-membered cyclic ring containing 0 to 3 heteroatoms selected from N, O and S. Specific types of aryl, heteroaryl, and heterocyclic ring are provided for in the definition of these different groups. In different embodiments, 0, 1 to 6 substituents, 2 to 6 substituents, 3 to 6 substituents, 4 to 6 substituents, 5 to 6 substituents, 6 substituents, 1 to 5 substituents, 2 to 5 substituents, 3 to 5 substituents, 4 to 5 substituents, 5 substituents, 1 to 4 substituents, 2 to 4 substituents, 3 to 4 substituents, 4 substituents, 1 to 3 substituents, 2 to 3 substituents, 3 substituents, 1 to 2 substituents, 2 substituents, or 1 substituent are present.

R⁸ is either an C₁-C₆ alkyl, H, C₁-C₃-heterocyclic ring.

Unless expressly stated to the contrary in a particular context, any of the various cyclic rings and ring systems described herein may be attached to the rest of the compound at any ring atom (i.e., any carbon atom or any heteroatom) provided that a stable compound results.

Unless expressly stated to the contrary, all ranges cited herein are inclusive. For example, a heteroaromatic ring described as containing from “1 to 4 heteroatoms” means the ring can contain 1, 2, 3 or 4 heteroatoms. It is also to be understood that any range cited herein includes within its scope all of the sub-ranges within that range. Thus, for example, a heterocyclic ring described as containing from “1 to 4 heteroatoms” is intended to include as aspects thereof, heterocyclic rings containing 2 to 4 heteroatoms, 3 or 4 heteroatoms, 1 to 3 heteroatoms, 2 or 3 heteroatoms, 1 or 2 heteroatoms, 1 heteroatom, 2 heteroatoms, 3 heteroatoms, and 4 heteroatoms.

When any variable occurs more than one time in any constituent or in Structure A or in any other formula depicting and describing compounds of the present invention, its definition on each occurrence is independent of its definition at every other occurrence. Also, combinations of substituents and/or variables are permissible only if such combinations result in stable compounds.

Unless expressly stated to the contrary, substitution by a named substituent is permitted on any atom in a ring (e.g., cycloalkyl, aryl, or heteroaryl) provided such ring substitution is chemically allowed and results in a stable compound.

As would be recognized by one of ordinary skill in the art, certain of the compounds of the present invention can exist as tautomers. All tautomeric forms of these compounds, whether isolated or in mixtures, are within the scope of the present invention. For example, in instances where a hydroxy (—OH) substituent is permitted on a heteroaromatic ring and keto-enol tautomerism is possible, it is understood that the substituent might in fact be present, in whole or in part, in the keto form.

A “stable” compound is a compound which can be prepared and isolated and whose structure and properties remain or can be caused to remain essentially unchanged for a period of time sufficient to allow use of the compound for the purposes described herein (e.g., therapeutic or prophylactic administration to a subject).

As a result of the selection of substituents and substituent patterns, certain compounds of the present invention can have asymmetric centers and can occur as mixtures of stereoisomers, or as individual diastereomers, or enantiomers. All isomeric forms of these compounds, whether individually or in mixtures, are within the scope of the present invention.

Examples of different embodiments for compounds of Structure A or pharmaceutically acceptable salts thereof are provided below. Variables not provided for in a particular embodiment are as originally defined or as provided for in another embodiment.

In a first embodiment, R₁ is an optionally substituted aryl; R₁ is an optionally substituted phenyl or napthyl; R₁ is phenyl substituted with 1-2 halogens; or R₁ is either 2,3 difluorophenyl, 3,4 dichlorophenyl, or 2-napthyl; and the other variables are provided for as originally defined.

In a second embodiment, R₂ is an optionally substituted aryl; R₂ is an optionally substituted phenyl; or an unsubstituted phenyl, and other variables are as originally defined or as provided for in the first embodiment. In a specific embodiment, when R₂ is an optionally substituted phenyl, R₁ is an optionally substituted aryl.

In a third embodiment, R₃ is methyl, ethyl, propyl or H; and other variables are as originally defined, or as provided for in the first or second embodiments.

In a fourth embodiment, R₄ is ethyl or H; and the other variables are as originally defined or as provided for in the first, second or third embodiments.

In a fifth embodiment, X is N, R₅ is H or C₁-C₆, and R₆ is either piperidine or

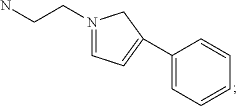

and the other variables are as provided for as originally defined or as provided for in the first, second, third, or fourth embodiments.

In a sixth embodiment, R₅ and R₆ together with X form an optionally substituted 5 to 6 membered heterocyclic ring; R₅ and R₆ together with X form an optionally substituted 5 to 6 membered heterocyclic ring containing 1 or 2 heteratoms, where each heteroatom is N; a 6 member heterocyclic ring with 2 N substituted by 1-2 methyls, a 6 member heterocyclic ring with 1 N substituted by methyl and substituted alkyl, a substituted 5 member homocyclic ring, a piperidine substituted by an alkyl piperidine or a piperidine substituted by a methyl. The other variables are as originally defined or as provided for in the first, second, third, or fifth embodiments.

Reference to the other variables as originally defined or as provided for in one or more prior embodiments indicates that variables not listed in a particular embodiment can be as indicated in structure A or as provided for in an indicated embodiment. Reference in an indicated embodiment to another embodiment can expressly be taken into account in determining the variables. For example, reference in the sixth embodiment to the fifth embodiment specifically allows for X, R₅ and R₆ as provided for in the fifth embodiment and, as indicated in the fifth embodiment, other variables can be as originally defined or as provided for in the first, second, third, or fourth embodiment,

### 5.3 Autoimmune Disorders

In another embodiment, the assay of the invention is used to identify small molecule compounds useful as therapeutics for autoimmune disorders. The antigen component of the assay is a self-antigen, a fragment, or conformational mimetic thereof. The antibody component of the assay is an autoantibody that is associated with a pathological condition including, but not limited to, myasthenia gravis, rheumatoid arthritis, lupus erythematosis, diabetes mellitus type 1, and multiple sclerosis.

Various combinatorial regimens can be considered depending on the particular disease target. Compounds identified as therapeutics for autoimmune disorders by methods of the invention can be co-administered with, e.g., corticosteroid drugs, NSAIDs, or immunosuppressants such as cyclophosphamide, methotrexate and azathioprine.

### 5.3.1 Class 1

Small molecules that decrease or inhibit the binding signal in the assay by binding to the antigen component and interfering with antibody binding are Class 1 compounds. Such compounds can be administered to act as antibody antagonists to decrease or prevent the autoantibody from binding to the self-antigen in vivo or as research tools.

Any method known in the art can be used to determine if a compound is a Class 1 compound including, but not limited to, surface plasmon resonance (e.g., a biacore assay), ELISA, and functional assays.

### 5.3.2 Class 2

Small molecules that decrease or inhibit the binding signal in the assay by binding to the antibody component and interfering with antigen binding are Class 2 compounds. When a mimotope (Class 2 compound) is administered to an individual under conditions that do not elicit an immune response, the mimotope can act as an antagonist and disrupt antigen-antibody binding. Because the autoantibody used in the assay as the isolating antibody is associated with the pathological condition, it is not desirable for the mimotopes to elicit endogenous production of similarly binding antibodies. As such, the mimotopes should not be conjugated to a carrier protein and should not be administered with an adjuvant.

Additionally, compounds can be used as research tools, e.g., to identify the presence of an autoantibody in a sample.

Any method known in the art can be used to determine if a compound is a Class 2 compound including, but not limited to, surface plasmon resonance (e.g., a biacore assay), ELISA, and functional assays.

### 5.4 Compound Derivatives

The invention also encompasses derivatives of Class 1 and Class 2 compounds. In one embodiment, a derivative is made in order to accommodate compound conjugation with a carrier protein. In a preferred embodiment, a 6-aminohexanoic acid—containing linker arm is added to the compound of interest for coupling to a carrier protein. In another embodiment, a derivative is made in order to improve a property of the compound including, but not limited to, affinity for antigen or antibody, pharmacokinetics, toxicology profiles, aqueous solubility, and conformational or isomeric constraint. In a specific embodiment, a class 1 compound identified by the methods of the invention is derivatized such that it has an IC₅₀ of 1 μM or lower in a functional assay (e.g., VERTTICAL assay).

The compounds can be co-crystallized bound to their target molecule (i.e., the antigen component or the antibody component of the assay). Alternatively, binding of the compounds can be modeled in silico using existing crystal structures of the isolating assay component (see, e.g., Luftig et al., 2006, Nat Struct Mol Biol 13:740 for crystallized complex of monoclonal antibody D5 bound to 5-helix). This data can aid in selection of the best sites for compound derivatization. In embodiments using derivatization to accommodate compound conjugation with a carrier protein, the site for derivatization should not interfere with the ability of the compound to bind to the assay component. In embodiments using derivatization to improve a property of the compound, the site for derivatization should alter the ability of the compound to bind to the assay component in some manner.

Derivatives of Class 1 and Class 2 compounds can be made using standard techniques known in the art. Enantiomers, diastereomers, isomers, and racemic mixtures of the compounds identified by methods of the invention are also encompassed by derivatives of the compounds.

### 5.5 Administration

Compounds identified by methods of the invention or derivatives thereof can be administered to subjects, including the general population or a subset thereof, in need of treatment (e.g., a patient suffering from an immune disorder or a patient suffering from or likely to suffer from an infection by a pathogen). The compounds of the present invention may also be administered in the form of pharmaceutically acceptable salts. The term “pharmaceutically acceptable salt” provides for a salt which possesses the effectiveness of the parent compound and which is not biologically or otherwise undesirable (e.g., is neither toxic nor otherwise deleterious to the recipient thereof). Suitable salts include acid addition salts which may, for example, be formed by mixing a solution of the compound of the present invention with a solution of a pharmaceutically acceptable acid such as hydrochloric acid, sulfuric acid, acetic acid, trifluoroacetic acid, or benzoic acid. Compounds of the invention that carry an acidic moiety can have suitable pharmaceutically acceptable salts that include alkali metal salts (e.g., sodium or potassium salts), alkaline earth metal salts (e.g., calcium or magnesium salts), and salts formed with suitable organic ligands such as quaternary ammonium salts. Also, in the case of an acid (—COOH) or alcohol group being present, pharmaceutically acceptable esters can be employed to modify the solubility or hydrolysis characteristics of the compound.

The compounds, derivatives or pharmaceutically acceptable salts thereof can be formulated and administered to a patient using the guidance provided herein along with techniques well known in the art. Guidelines for pharmaceutical administration in general are provided in, for example, Vaccines Eds. Plotkin and Orenstein, W.B. Sanders Company, 1999; Remington's Pharmaceutical Sciences 20^{th} Edition, Ed. Gennaro, Mack Publishing, 2000; and Modern Pharmaceutics 2^{nd} Edition, Eds. Banker and Rhodes, Marcel Dekker, Inc., 1990.

Pharmaceutically acceptable carriers facilitate storage and administration of a compound, derivative or pharmaceutically acceptable salt thereof to a patient. Pharmaceutically acceptable carriers may contain different components such as a buffer, sterile water for injection, normal saline or phosphate buffered saline, sucrose, histidine, salts and polysorbate. The composition, if desired, can also contain minor amounts of wetting or emulsifying agents, or pH buffering agents. These compositions can take the form of solutions, suspensions, emulsion, tablets, pills, capsules, powders, sustained-release formulations and the like.

Suitable dosing regimens are preferably determined taking into account factors well known in the art including age, weight, sex and medical condition of the patient; the route of administration; the desired effect; and the particular compound employed.

Various delivery systems are known and can be used to administer a compound identified by methods of the invention or derivative or pharmaceutically acceptable salt thereof including, but not limited to, parenteral administration (e.g., intradermal, intramuscular, intraperitoneal, intravenous and subcutaneous), epidural, and mucosal (e.g., intranasal, inhaled, and oral routes). The compositions may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local.

The compositions can be delivered in a controlled release or sustained release system. In one embodiment, a pump may be used to achieve controlled or sustained release (see Langer, supra; Sefton, 1987, CRC Crit. Ref. Biomed. Eng. 14:20; Buchwald et al., 1980, Surgery 88:507; Saudek et al., 1989, N. Engl. J. Med. 321:574). In another embodiment, polymeric materials can be used to achieve controlled or sustained release of the compounds or derivatives thereof identified by the methods of the invention (see e.g., Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, New York (1984); Ranger and Peppas, 1983, J. Macromol. Sci. Rev. Macromol. Chem. 23:61; see also Levy et al., 1985, Science 228:190; Duringetal., 1989, Ann. Neurol. 25:351; Howard et al., 1989, J. Neurosurg. 71:105); U.S. Pat. Nos. 5,679,377; 5,916,597; 5,912,015; 5,989,463; 5,128,326; International Publication Nos. WO 99/15154 and WO 99/20253. Examples of polymers used in sustained release formulations include, but are not limited to, poly(2-hydroxy ethyl methacrylate), poly(methyl methacrylate), poly(acrylic acid), poly(ethylene-co-vinyl acetate), poly(methacrylic acid), polyglycolides (PLG), polyanhydrides, poly(N-vinyl pyrrolidone), poly(vinyl alcohol), polyacrylamide, poly(ethylene glycol), polylactides (PLA), poly(lactide-co-glycolides) (PLGA), and polyorthoesters. In a preferred embodiment, the polymer used in a sustained release formulation is inert, free of leachable impurities, stable on storage, sterile, and biodegradable. In yet another embodiment, a controlled or sustained release system can be placed in proximity of the prophylactic or therapeutic target, thus requiring only a fraction of the systemic dose (see, e.g., Goodson, in Medical Applications of Controlled Release, supra, vol. 2, pp. 115-138 (1984)). Controlled release systems are discussed in the review by Langer (1990, Science 249:1527-1533).

For compounds, derivatives or pharmaceutically acceptable salts thereof that are to be used as immunogens (see, e.g., Section 5.2.2), the timing of doses depends upon factors well known in the art. After the initial administration one or more booster doses may subsequently be administered to maintain or boost antibody titers. An example of a dosing regime would be day 1, 1 month, a third dose at either 4, 6 or 12 months, and additional booster doses at distant times as needed.

Additionally, immunogen compositions preferable contain one or more adjuvants. Adjuvants are substances that assist an immunogen in producing an immune response. An immunogen can be administered in conjunction with one or more adjuvants, wherein the adjuvants are mixed (before or simultaneously upon injection) with the immunogen. Alternatively the adjuvant is not mixed with the immunogen composition but is separately co-administered with the immunogen. Adjuvants can function by different mechanisms such as one or more of the following: increasing the antigen's biologic or immunologic half-life; improving antigen delivery to antigen-presenting cells; improving antigen processing and presentation by antigen-presenting cells; and inducing production of immunomodulatory cytokines (Vogel, 2000, Clinical Infectious Diseases 30 (suppl. 3) S266-270).

A variety of different types of adjuvants can be employed to assist in the production of an immune response. Examples of particular adjuvants include aluminum hydroxide (e.g., ALHYDROGEL®, REHYDRAGEL®), aluminum phosphate, aluminum hydroxyphosphate (e.g., ADJU-PHOS®), amorphous aluminum hydroxyphosphate (e.g., Merck Aluminum Adjuvant), or other salts of aluminum, calcium phosphate, DNA CpG motifs, monophosphoryl lipid A, cholera toxin, E. coli heat-labile toxin, pertussis toxin, muramyl dipeptide, muramyl dipeptide derivatives (e.g., N-acetyl-muramyl-L-threonyl-D-isoglutamine, threonyl-MDP, GMDP, N-acetyl-nor-muramyl-L-alanyl-D-isoglutamine, N-acetylmuramyl-L-alanyl-D-isoglutaminyl-L-alanine-2-(1′-2′-dipalmitoyl-sn-glycero-3-hydroxyphosphoryloxy)-ethylamine, muramyl tripeptide phosphatidylethanolamine (MTP-PE)), muramyl peptide analogues, Freund's incomplete adjuvant, MF59, MF59C, MF59C.1, SAF, immunostimulatory complexes, liposomes, biodegradable microspheres, saponins (e.g., QuilA, QS-21, ISCOMATRIX®), nonionic block copolymers (e.g., L-121 (polyoxypropylene/polyoxyethylene), homo- and copolymers of lactic acid (PLA) and glycolic acid (PGA), poly(lactide-co-glycolides) (PLGA) microparticles, polyphosphazene, synthetic polynucleotides, cytokines (e.g., interleukin-2, interleukin-7, interleukin-12, granulocyte-macrophage colony stimulating factor (GM-CSF), interferon-γ, interleukin-1β, and IL-1β peptide or Sclavo Peptide), cytokine-containing liposomes, triterpenoid glycosides, heat-labile enterotoxin from Escherichia coli (LT), cholera holotoxin (CT) and cholera Toxin B Subunit (CTB) from Vibrio cholerae, mutant toxins (e.g., LTK63 and LTR72), ISCOMS and Toll-like receptor agonists (Vogel, 2000, Clinical Infectious Diseases 30(suppl 3): S266-270; Klein et al., 2000, Journal of Pharmaceutical Sciences 89:311-321; Rimmelzwaan et al., 2001, Vaccine 19:1180-1187; Kersten, 2003, Vaccine 21:915-920; O'Hagen, 2001, Curr. Drug Target Infect. Disord. 1:273-286).

The contents of all published articles, books, reference manuals and abstracts cited herein, are hereby incorporated by reference in their entirety to more fully describe the state of the art to which the invention pertains.

As various changes can be made in the above-described subject matter without departing from the scope and spirit of the present invention, it is intended that all subject matter contained in the above description, or defined in the appended claims, be interpreted as descriptive and illustrative of the present invention. Modifications and variations of the present invention are possible in light of the above teachings.

### 6. EXAMPLES

### 6.1 DCBA Assay

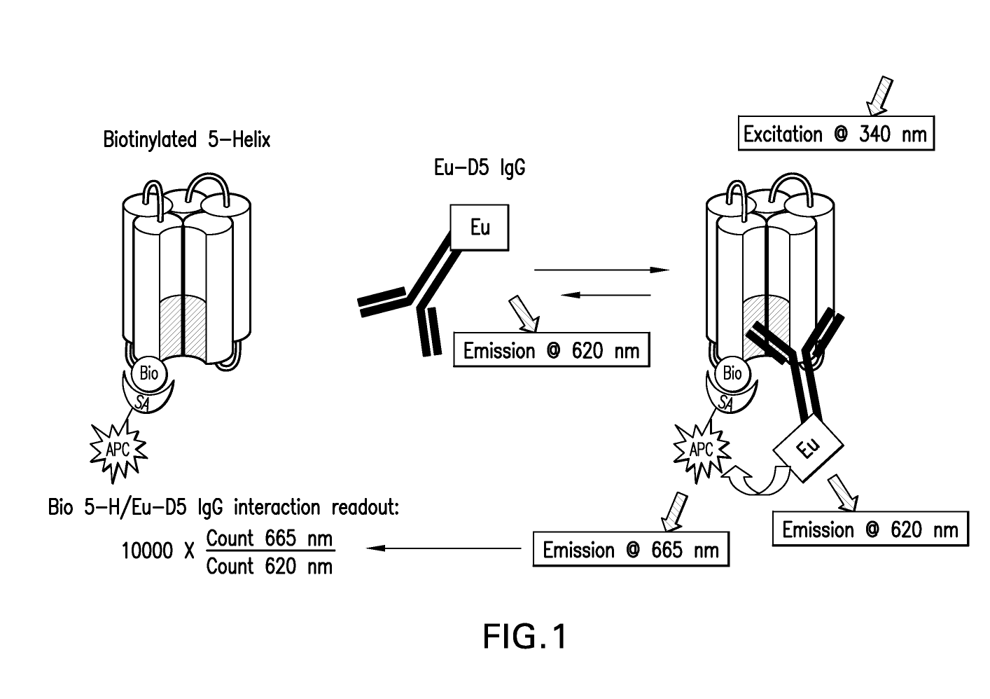

The D5 Competitive Binding Assay (DCBA) used as a basis for high throughput screening is schematically outlined in FIG. 1. Monoclonal antibody D5 specifically recognizes and binds to a well-characterized hydrophobic pocket contained within the HR1 domain of HIV-1 envelope protein gp41. This binding blocks the intramolecular folding of gp41 into a six-helix bundle structure that is essential for membrane fusion and viral entry. An in vitro binding assay was developed using D5 IgG conjugated to europium cryptate (Eu-D5) and a biotinylated gp41 mimetic molecule that presents the hydrophobic pocket in a stabilized structural context (termed 5-helix (or 5H) peptide). The assay readout is based on a fluorescence resonance energy transfer (FRET) format. Biotin-5-helix binds to a streptavidin-conjugated allophycocyanin (APC) substrate to form a 5H-SA-APC complex. When Eu-D5 binds to the hydrophobic pocket of biotin-5H, it brings the Eu into close proximity with the APC substrate to allow FRET from Eu to APC. When the reaction system is excited at a wavelength of 340 nm, the Eu signals at 620 nm. If the Eu is in close proximity to the APC, it excites the APC which results in long-lived emissions at 665 nm. Introducing a time delay (e.g., ˜100 μseconds) between the system excitation and fluorescence measurement allows the FRET signals to be cleared of non-specific short-lived emissions (“time-resolved FRET”). The resulting specific FRET signal indicative of the amount of Eu-D5 bound to biotin-5H can be quantified by the ratio of fluorescence [counts at 665 nm/fluorescence counts at 620 nm]×10,000. Agents that interfere with this binding (through competitive binding to either D5 or 5-helix) will cause a decrease in the ratio value.

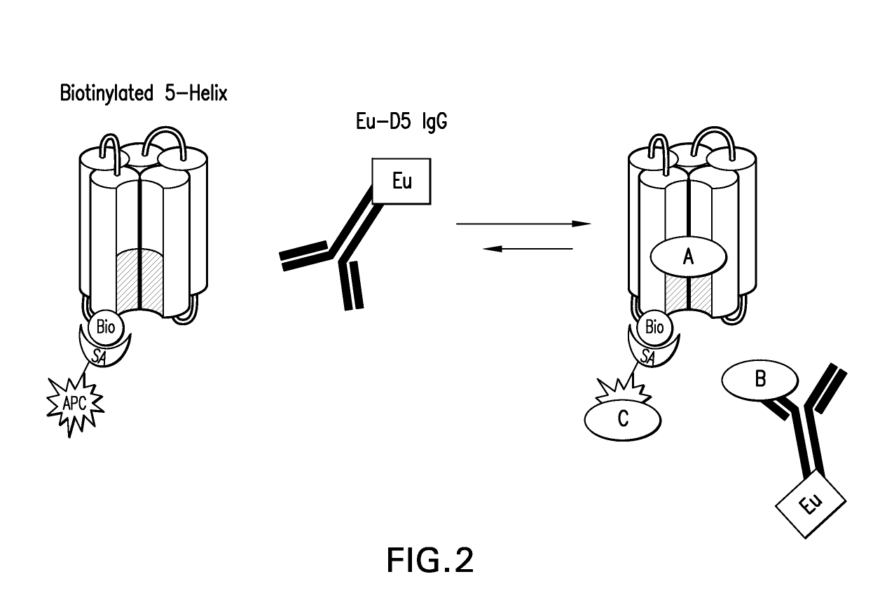

FIG. 2 shows the three classes of compounds which can inhibit binding of D5 to 5-helix in this assay: (1) molecules which bind to the hydrophobic pocket on 5-helix, (2) molecules which bind to the CDR region of D5, and (3) molecules which can interfere with the assay readout, e.g., through interaction with any component of the biotin-streptavidin-allophycocyanin complex or with europium cryptate and thus inhibit effective energy transfer from the Eu label. This last group represents non-specific, non-mechanistic binding antagonists.

Briefly, the assay was performed in the following manner. To each well of a 96-well plate, 20 μl of 1× LANCE detection buffer (PerkinElmer CR97-100), 20 μl of 25 nM SA-APC (PerkinElmer AD0201), 20 μl of 20 nM biotin-5-helix, and candidate binding agent (i.e., antibodies/compounds to be tested or a serial-diluted positive control of known competing agents such as D5 or C34) was added. The mixture was kept in the dark at room temperature for 30 min. Twenty (20) μl of 10 nM Eu-D5 was subsequently added into each well and the plate was kept in dark at room temperature overnight. The plate was then read using the Fusion Universal Microplate Analyzer (Packard Bioscience). The ratio value of [counts 665 nm/counts 620 nm]×10,000 was plotted as a function of the concentration of each positive control or test compound/antibody in order to calculate its IC₅₀ (data not shown).

### 6.2 Adaptation of DCBA Assay for High Throughput Screening

Because the binding affinity of the D5 antibody to 5-helix needed to be considered when screening for inhibitors, concentrations of D5 and 5-Helix were maintained in the miniaturized format to be below their Kd values. The assay volumes were adjusted to accommodate high-throughput screening in a miniaturized format and achieve the desired compound screening concentration. The final assay volume was reduced to 2.5 uL and component concentrations adjusted so that 2 ul of the biotinylated-5-helix protein could be added, followed by compounds in DMSO and finally followed by 0.5 ul of D5-Eu and Streptavidin labeled with Allophycocyanin (APC). The conditions were then validated by their ability to be inhibited with the peptide D10-p5-2k and with unlabeled D5 antibody.

### 6.3 Identification of Non-Specific Inhibitors in the Compounds Positive in the DCBA Assay

A compound collection was screened using the DCBA assay as a primary screen and a modified version of the assay which employed a Eu-labeled non-D5 monoclonal antibody as the counter-screen. In order to identify compounds that gave positive results in the assay as in Class 1 (compounds that bind to the hydrophobic pocket of 5-helix), Class 2 (compounds that bind to the CDR region of D5) or Class 3 (compounds that can interfere with the assay readout through interaction with any component of the bioton-streptavidin-allophycocyanin complex or with europium cryptate), a counter-screen based on monoclonal antibody F19 was employed. F19 is an antibody which binds to one of the scaffolding outer helical bundles on 5-helix in close enough proximity to effect an energy transfer event to APC when labeled with Eu. This is shown diagrammatically in FIG. 3. Compounds which were positive in both the DCBA primary and F19 counter screens were regarded as non-specific inhibitors of the Eu-APC energy transfer event.

### 6.4 Results of Automated High Throughput Screening of DCBA Assay

For the primary screen, an inhibition cut-off value of 31% was employed, along with the following filter criteria: (1) elimination of biotin-containing compounds, (2) elimination of compounds with undefined side chains (structures containing generic “R” or “X” groups), and (3) elimination of any compounds which scored in >5 unrelated screens. The number of positive compounds identified after application of the filters was 5,679. Two inhibition thresholds were used to score a compound as positive following F19 counter-screening: (1) >25% D5 inhibition and <20% F19 inhibition and (2) D5 inhibition >F19 inhibition+20%. Using the more stringent filter (1), 120 hits were identified while 154 hits were found using filter (2). An analysis of the data showed that 50% of all the positive compounds could be grouped into 4 general structural classes (one class is represented by Compound 1.1 in Table 2). Of the compounds listed in Table 2, Compound 1.1 was the best in terms of specific inhibition of D5 binding relative to F19 inhibition.

### 6.5 Identification of Class 1 Compounds by Antiviral Activity Screening

The Viral Entry, Reverse Transcription, and Integration: Cellular Assay for Leads (VERTICAL) was conducted to identify positive DCBA lead compounds which bind to the hydrophobic pocket region of 5-helix and act to inhibit viral entry.

P4/R5 cells (HeLa cells expressing endogenous CXCR4 and stably transfected to express CD4 and CCR5 which also contain an integrated β-galactosidase reporter gene under control of an HIV LTR promoter) were maintained in phenol red-free Dulbecco's modified Eagle's medium, 10% fetal bovine serum, 1% penicillin/streptomycin and are seeded in 96-well plates at 2.5×10³ cells/well. Cells were infected the day after plating with the HXB2 strain of HIV-1 (CXCR-4 using clinical isolate obtained from Advanced Biotechnology Inc., Bethesda, Md.) in the presence of titrations of DCBA lead compounds. Cells with and without virus addition were used to establish maximal and minimal infectivity signals, respectively. After 48 h of infection, cells were lysed, and the β-galactosidase activity (which is indicative of the viral replication level) was detected by a Dynex luminometer using Gal Screen™ chemiluminescent substrate (Applied Biosystems, Foster City, Calif.). Although decreased β-galactosidase activity was observed in the presence of some of the compounds, the reason for the decrease was not certain (e.g., compounds blocking any step of the early HIV life cycle including entry, reverse transcription, integration, and tat-mediated transcription can all inhibit production of β-galactosidase and lead to decreased viral signals).

In order to determine whether the DCBA lead compounds that decrease β-galactosidase activity specifically block the entry step, the VERTICAL assay was conducted using an HIV-1 virus pseudo-typed with the VSV-G envelope protein. This virus differs from native HIV-1 only at the entry step as VSV-G takes a different entry route as HIV-1. Specific HIV-1 gp41 binding compounds (both Class 1 and Class 2) would not be expected to inhibit the entry of VSV-G pseudo-typed HIV-1 (VSV-G).

The compounds which scored positive following primary DCBA screen and F19 counter-screen (n=140) were tested in a 1^{st} round VERTICAL analysis for their ability to neutralize viral entry using wild type HXB2 virus and VSV-G. Any compounds which inhibited entry of both HXB2 and VSV-G were likely not acting by a gp41-specific mechanism. This analysis identified 4 compounds which were shown to specifically inhibit entry of HXB2 but not VSV-G. In order to further demonstrate that these compounds were targeting the hydrophobic pocket region of gp41, they were tested in a second round which included a repeat with HXB2 and VSV-G, along with 3 additional viruses which contained single point mutations within the hydrophobic pocket region of gp41 (L568A, V570A, K574A). All four compounds showed antiviral activity with IC₅₀ values of approximately 10 uM against HXB2, but no activity against either VSV-G or any of the pocket-mutated strains.

### 6.6 Identification of Class 2 Compounds by Surface Plasmon Resonance Screening

Surface Plasmon Resonance (SPR) was conducted using a Biacore A100 instrument to identify positive DCBA lead compounds which bind to the D5 antibody and may represent mimotopes of the gp41 hydrophobic pocket region. Negative controls of non-relevant isotype-matched IgG1 and 6-helix were used. 6-helix is a peptide similar to 5-helix except that the C-peptide binding site is occupied by an attached C-peptide (i.e., all six helices that constitute the gp41 trimer-of-hairpins have been linked into a single polypeptide) obscuring the hydrophobic pocket.

### 6.6.1 Protein Immobilization and Assay Protocol

Immobilization of D5, 5-helix, 6-helix and IgG1 was performed using amine coupling to a research grade carboxymethylated dextran chip (CM5) Amine coupling was accomplished by first activating the chip surface with a 10 minute injection of EDC/NH S followed by a 10 minute injection of each protein diluted to 10 ug/ml (20 ug/ml for 5-helix) in 10 mM Sodium Acetate pH 5. One hundred and fifty nine (159) compounds were tested at 20 uM and 2 uM in a running buffer of 10 mM HEPES, 150 mM NaCl, 3 mM EDTA, 0.05% p20 and 1% DMSO. Contact time was 120 sec, with a dissociation time of 240 sec and a flow rate of 30 ul/min, 25 C. Regeneration conditions were 1M NaCl diluted in running buffer injected for two 45 sec pulses. The data collected was analyzed using Biacore's Evaluation Screening program, all data was DMSO solvent corrected.

### 6.6.2 Hit Selection

The compounds that were the most D5-specific binders were identified by analyzing binding rate responses. A measurement of maximum binding level (Rₘₐₓ) was calculated for each of the proteins to be analyzed by binding to the compounds (i.e., 5-helix, 6-helix, IgG1, and D5). (Rmax=(Compound MW/Protein MW)×level of protein immobilized×stoichiometry). Compounds that gave a response which was ≧10% of the protein's calculated Rₘₐₓ were defined as binders. Using Excel, a score of 1 was assigned to compounds that passed the cut-off for each protein analyzed. The scores helped to identify non-specific and non-binders because the specific binders should get an overall score of 1 and the non-binders a score of 0. Since there were no specific 5-helix binders using this protocol, compounds binding D5 were sorted by binding level at 2 uM and the top 64 compounds were chosen for titration and kinetic analysis. Methods known in the art were used to perform titration and kinetic analysis. Compound 2 was shown to have a fast on/slow off rate while Compound 3 has a slow on/fast off rate.

### 6.7 Derivatives of Class 2 Compounds

Class 2 compounds are modified to make derivative compounds. The derivatives may have altered properties in comparison to the unmodified compound. Examples of Class 2 derivatives that were synthesized are shown in Table 3 (see Section 6.13).

Modified compounds can be tested to determine if the modification alters the ability of the compound to bind to D5 as compared to the unmodified compound. In one embodiment, the Biacore assay described in Section 6.6 can be used to determine the ability to bind to D5.

### 6.8 Attachment of Linkers to Class 2 Compounds

To render a small molecule hapten immunogenic, it must first be covalently coupled with a carrier protein in order to provide T cell epitopes for the immunogen. The haptens must be derivatized in such a way as to preserve the ability to bind to the antibody while providing a linker to the protein carrier. To identify putative points of contact between D5 and the Class 2 compounds, molecular modeling was performed using the published crystal structure of D5 bound to 5-helix (Luftig et al., 2006, Nat Struct Mol Biol 13:740-7). The epitope for D5 antibody binding lies in the hydrophobic pocket region located near the carboxy terminal half of the HR1 trimer. Amino acids Leu 568, Trp 571 and Lys 574 of gp41 are critical for antibody binding whereas Val 570 contributes to a lesser extent.

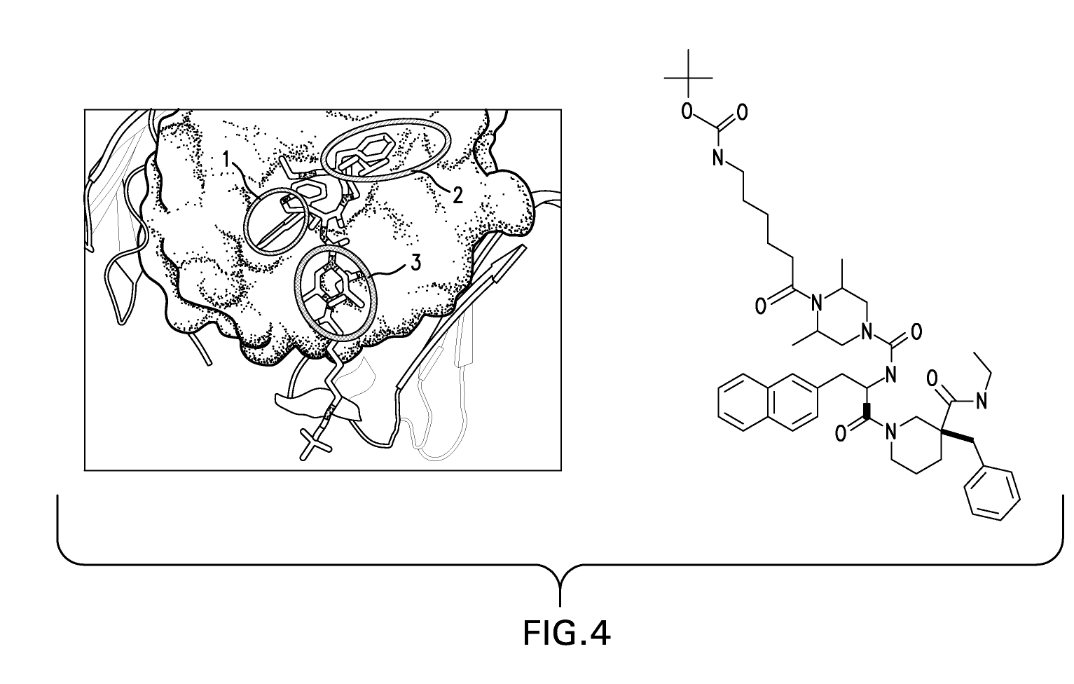

FIG. 4 shows Compound 2.4 with an Aha linker attached overlaid on the three 5H residues that make critical contacts in the CDR pocket of the antibody. Importantly, the derivatized part of the molecule containing the amino hexanoic acid (Aha) spacer and the boc protective group are pointing away from the contact residues of the antibody combining site. Binding of boc-Aha derivatized Compound 2.4 to D5 was confirmed by SPR assay with no loss of affinity. Groups on the hapten which mimic amino acids on 5-helix which make critical contacts within the D5 CDR are circled (circle 1 shows that naphthyl mimics Trp 571 on 5-helix; circle 2 shows that benzyl mimics Leu 568 on 5-helix; circle 3 shows that urea mimics Gln 575 on 5-helix).

Below is a schematic compound depicting possible sites for attachment of the linker. The R variables in the schematic compound are such that the compound falls within the genus of compounds disclosed in Section 5.2.3 for Structure A. M1 through M4 were positions identified on the substituted piperidine series which were amenable to additional derivatization. Derivatization with Aha at M1 did not affect the binding of haptens to D5. Accordingly, this area was used to attach the linker to the compounds (including the Aha that was attached to Compound 2.4).

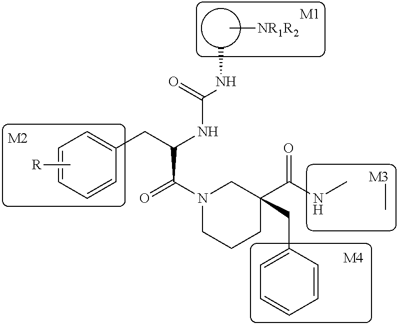

Schemes 1 and 2 show examples of addition of an Aha liner to Compound 1.2 (see Table 2) in preparation of conjugation to a carrier protein. In Scheme 1, the Aha linker is added in a 2-step process while in Scheme 2 a pre-formed Aha linker was added. Also, the thiol group introduced in the linker is from a thioacetyl in Scheme 1 rather than a cysteine residue as in Scheme 2. Conjugation of Compounds 2.45 and 2.58 (see Section 6.9) was done according to the chemistry as shown in Scheme 2.

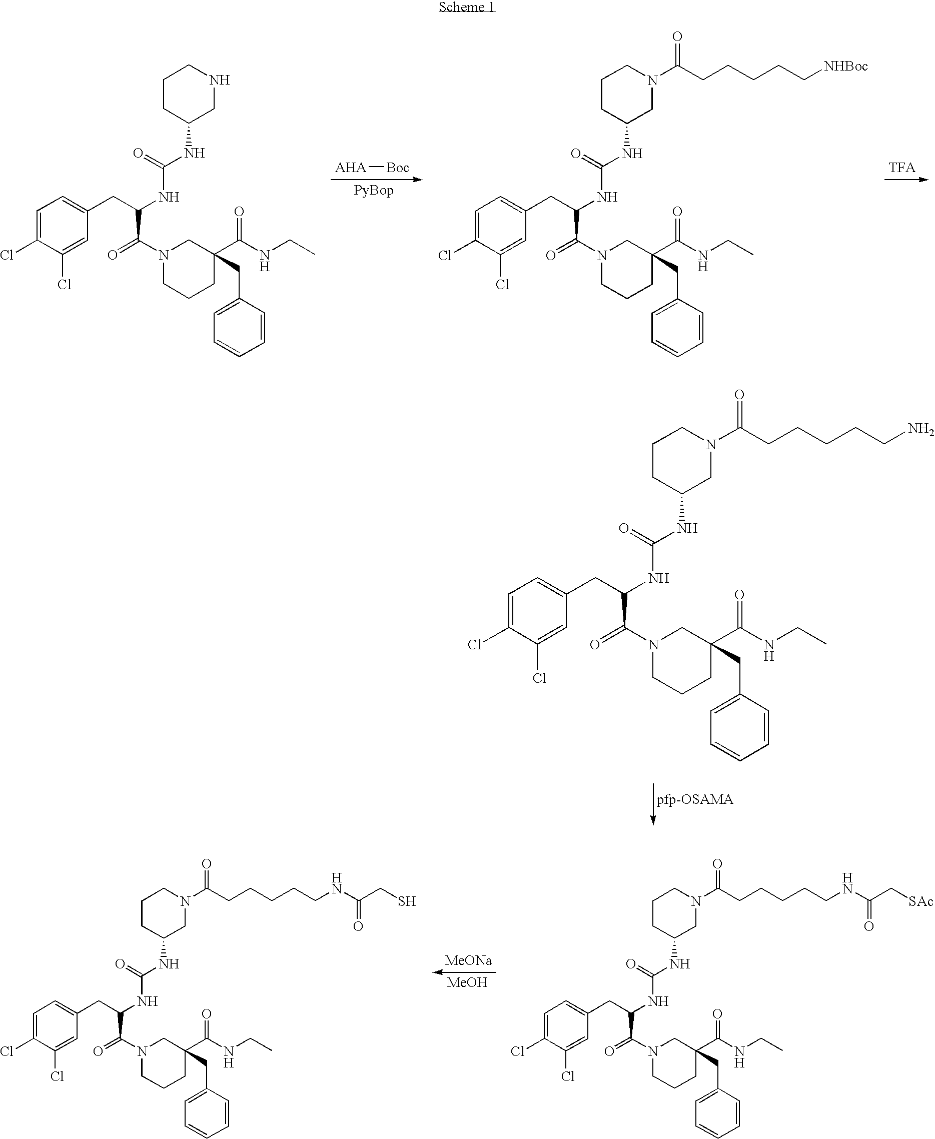

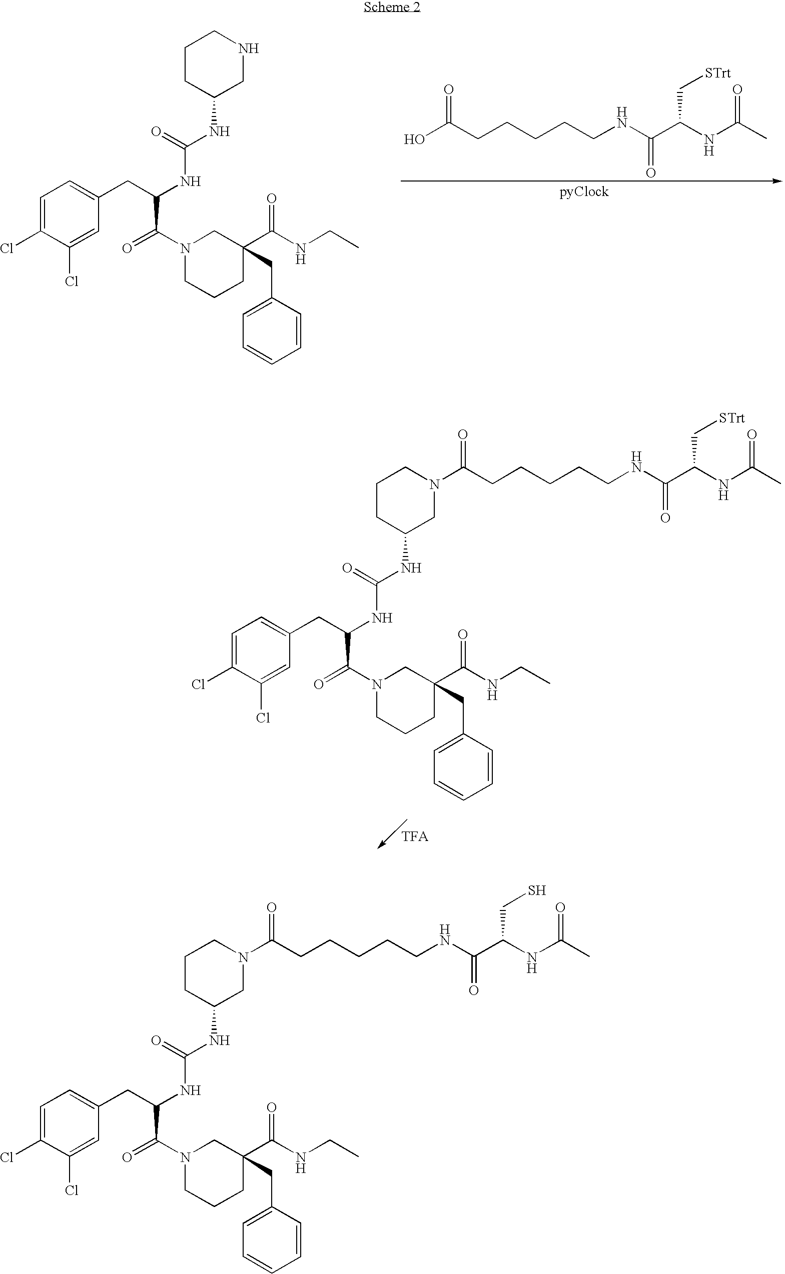

### 6.9 Conjugation of Class 2 Compounds to Carrier Proteins

The carrier protein used to conjugate to compounds of the invention was CRM197 (a mutant diphtheria toxin; see Steinhoff et al., 1994, Pediatr Infect Dis J 13: 368-72). CRM197 was dissolved at 1 mg/ml in 25 mM HEPES, pH 7.3, 0.15M sodium chloride, 5 mM ethylenediaminetetraacetic acid. It was then maleimidated via a portion of its surface accessible lysine residues by reaction with a 10-fold molar excess of SMCC (Succinimidyl 4-[N-maleimidomethyl]cyclohexane-1-carboxylate): TNBS (2,4,6-trinitrobenzenesulfonic acid) accessible lysine residues for 3 hr at 22° C. The maleimidated protein was purified from reaction components by desalting on a GE Biosciences HiPrep 26/10 column equilibrated in HBS/EDTA. Maleimide incorporation was quantified using a thiol-consumption assay. The average derivatization was 139 nmole maleimide per mg CRM197.

The DCBA hapten, Compound 2.45 or Compound 2.58, was dissolved in ethanol at a concentration of 10 mg/ml. The compound was mixed with maleimidated CRM197 (0.25 mg/ml) at a 3:1 molar ratio of thiol:maleimide in HBS/EDTA containing 10% ethanol. The conjugation reaction was allowed to proceed at 22° C. for 2 hours at which time precipitated protein was removed by centrifugation. The clarified supernatant was dialyzed at 22° C. against 3 changes of 4 L 25 mM HEPES, pH 7.3, 0.15M sodium chloride over a period of 24 hours. The dialyzed conjugate was then concentrated approximately 4-fold over a 30 kDa molecular weight cut-off membrane.

Conjugation efficiency was determined by amino acid analysis for quantitation of 6-aminohexanoic acid (a component of the linker region of the hapten), and S-dicarboxyethylcysteine (a unique residue generated by the formation of a covalent bond between hapten and carrier). For example, when Compound 2.45 was conjugated to CRM197, the Aha/CRM ratio was 33; the SDCEC/CRM ratio was 15 giving a concentration of conjugate of 642 ug/ml.

Conjugates were formulated for animal studies by adsorption to Merck aluminum adjuvant. This was accomplished by mixing an appropriate volume of conjugate with an equal volume of 2× aluminum adjuvant at room temperature for 30 minutes. Completeness of adsorption was determined using a commercial BCA protein assay to quantify unabsorbed protein in the vaccine supernatant.

### 6.10 Induction of an Immune Response by Class 2 Compounds

Conjugates were made with two DCBA haptens, Compound 2.45 and Compound 2.58 (see Table 3 and Table 4, Form A), and tested for immunogenicity in mice. Three to six week-old female balb/c mice (Taconic, Hudson, N.Y.) were maintained in the animal facilities of Merck Research laboratories in accordance with institutional guidelines. All animal experiments were approved by Merck Research Laboratories Institutional Animal Care and Use Committee (IACUC). Class 2 conjugates were formulated with 450 μg of Merck aluminum alum and 1 mg of IMO-2055 (Idera Pharmaceuticals, Inc Cambridge, Mass.) per ml in PBS. Mice of 10 per group were immunized intramuscularly with 100 μl of the vaccine containing 25 μg of conjugate. The immunizations were carried out three times at 2-week intervals. Serum samples, obtained from tail vein venipuncture, were collected in Microtainer® Serum Separator Tubes (BD, Franklin Lakes, N.J.), two weeks post dose 2 and 3, at weeks four, six and eight. Serum samples were stored at 4° C. until use.

Binding activity of mouse antisera was carried out by enzyme-linked immunosorbent assay (ELISA). Ninety-six well plates (Maxisorb Nunc) were coated with 50 μl per well of various test antigen, including: biotinylated self antigen (Class 2 compound used as immunogen), 5-Helix and synthetically derived gp41 hydrophobic pocket N peptides ccIZN17 and ccINZ36 (constrained constructs that contain the hydrophobic pocket of 5-Helix; see Bianchi et al., 2005, PNAS 102: 12903-8). Similarly, a peptide substrate was utilized to discriminate any anti-linker cross reactivity, via a non-HIV related biotinylated peptide from Influenza. In addition, mouse antiserum was tested against the carrier protein CRM-197. Moreover, known positive antiserum, generated separately was used to validate all coating substrates. Each substrate was coated at a concentration of 4 mcg/μl, overnight at 4° C. Plates were washed six times with PBS containing 0.05% Tween-20 (PBST) and blocked with 3% skim milk in PBST (milk-PBST). Mouse test antiserum was prepared in milk-PBST starting at 1:100 dilution followed by serial 4-fold dilutions. 100 μl diluted anti-sera were added to each well, and the plates were incubated for 2 hr at room temperature, which was followed by three washes with PBST. Fifty microliters of HRP-conjugated goat anti-mouse IgG secondary antibody (Invitrogen, Inc., Carlsbad, Calif.) at 1:5000 dilution in milk-PBST was added per well and incubated at room temperature for 1 hr. Plates were washed six times followed by addition of 100 μl per well of 3,3′,5,5′-tetramethylbenzidine (TMB) (Virolabs, Chantilly, Va.). After 3-5 min incubation at room temperature the reaction was stopped by adding 100 μl of stop solution (Virolabs, Chantilly, Va.) per well. Plates were read at 450 nm in a microplate reader. Titers were determined by the reciprocal of the dilution that was above background plus two sigma. Results are expressed as the geometric mean of reciprocal endpoint titers with titers <100 being assigned a value of 100 for the purpose of calculations.

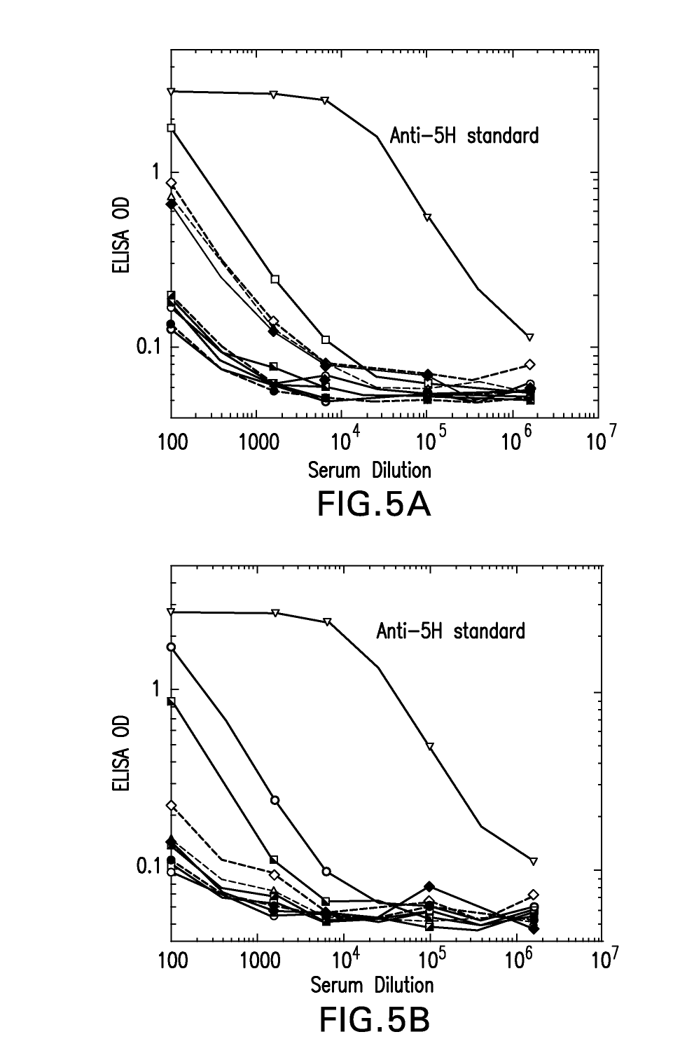

The results shown in Table 1 indicate that the DCBA hapten conjugates elicited very high titered antibodies to the haptens and that the antisera crossreacted strongly to each hapten. However, the hapten conjugates elicited only weak antibody responses to 5H and even weaker responses to ccIZN17. Although most mouse sera had little or no activity in the 5H ELISA, some individual sera did have more pronounced binding activity elicited by Compound 2.45 (FIG. 5A) and Compound 2.58 (FIG. 5B).

TABLE 1

| | Post-dose 3 geometric mean | | | | | |
|---|---|---|---|---|---|---|
| | antibody titer (GMT) elicited versus: | | | | | |
| Immunogen | Compound | Compound | | | | |
| Used | 2.45 | 2.58 | 5-helix | ccIZN17 | ccIZN36 | CRM₁₉₇ |
| Compound | >1,600,000 | >1,600,000 | 400 | <100 | <100 | 1,600 |
| 2.45 | | | | | | |
| Compound | >1,600,000 | 713,155 | 200 | 200 | <100 | 400 |
| 2.58 | | | | | | |
| ccIZN17 | 100 | 152 | 1,393 | 102,400 | 102,400 | <100 |

Sera can be assayed for neutralization efficiency, e.g., using a VERTICAL assay. Conjugates that produce a neutralizing immune response are administered to monkeys, e.g., rhesus macaques, before challenge of the monkeys with SHIV. The monkeys are examined for a protective effect induced by the conjugate vaccination.

### 6.11 Synthesis of Compounds of the Invention

The compounds encompassed by Structure A can be made using guidance provided herein and the techniques known in the art. The R variables in the compounds shown in the Schemes are such that the compounds of the Schemes fall within the genus of compounds disclosed in Section 5.2.3 for Structure A. The compounds in Table 2 were made using methods described in Schemes 3 and 4 and exemplified in Schemes 5 and 6.

In one specific embodiment, Scheme 3, the starting carboxylic acid A1 (Yang et al, 1998, J. Med. Chem. 41:2439-2441) can be coupled with amine R²NH using a variety of amide-bond forming techniques to provide Boc-protected A2, which is then deprotected under acidic conditions to give free substituted piperidine A3. Coupling of A3 and protected amino acid A4 gives A5, and then, after another Boc deprotection, secondary amine A6. This amine can be again acylated with another protected amino acid to give A7 and then deprotected A8. Finally, amine A8 can be further coupled with an amino acid R⁶COOH to provide A9.

In another specific embodiment, Scheme 4, amine A6 can be converted into asymmetric urea B2 through the intermediacy of succinimidyl derivative B1 formed by treating A6 with N,N′-disuccinimidyl carbonate. B2 can then be deprotected to give amine B3, which is acylated to furnish B4.

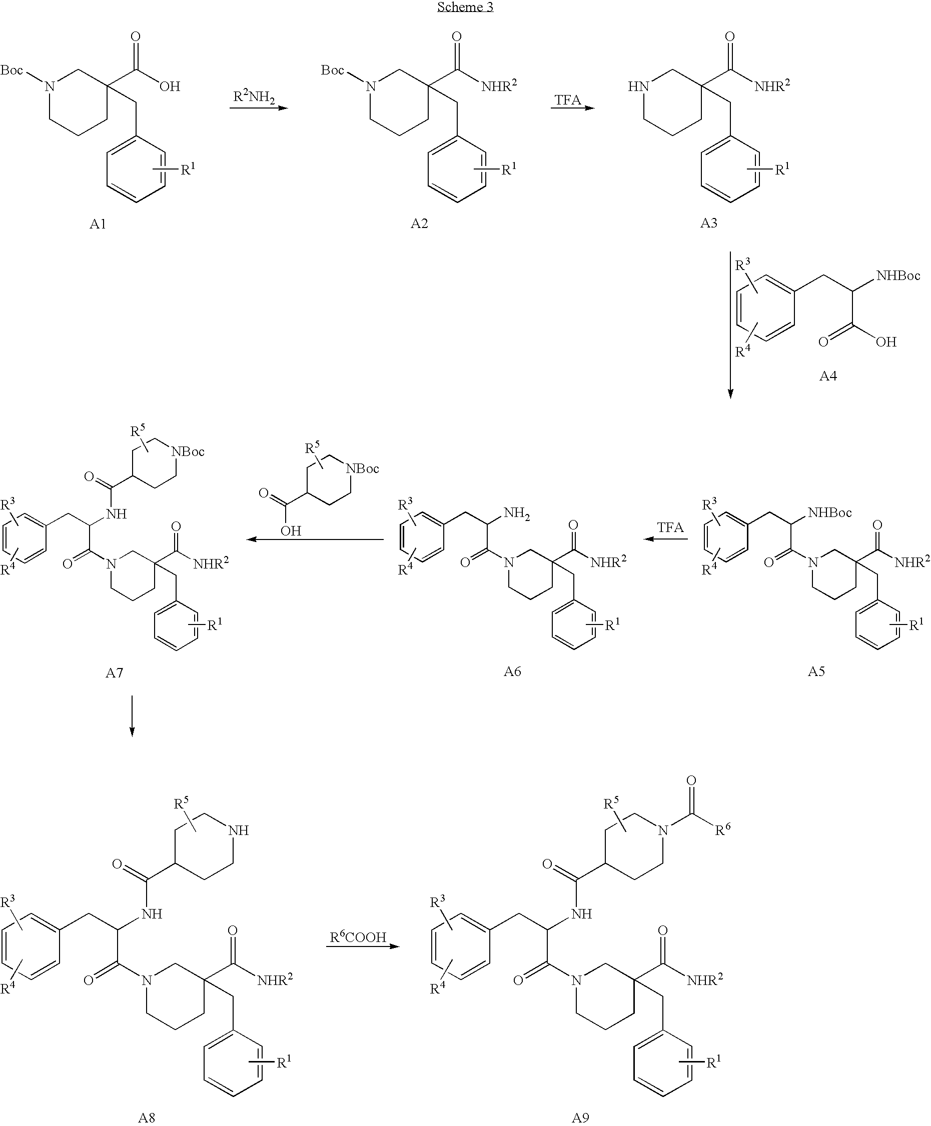

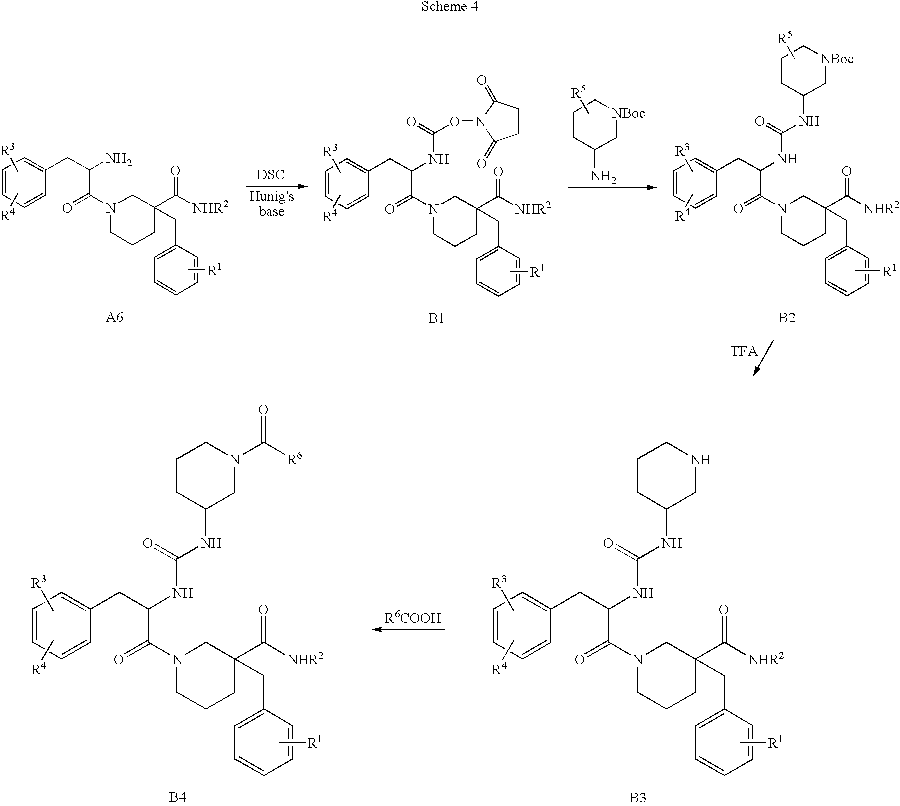

### 6.12 Synthesis of Compounds Identified by DCBA Screen

The compounds in Tables 1 and 2 can be made using guidance provided herein and the techniques known in the art. Preparation of compounds 1.1 (compound 15 in Scheme 6), 1.8 (compound 10 in Scheme 5) and 2.8 (compound 17 in Scheme 6) are described as examples.

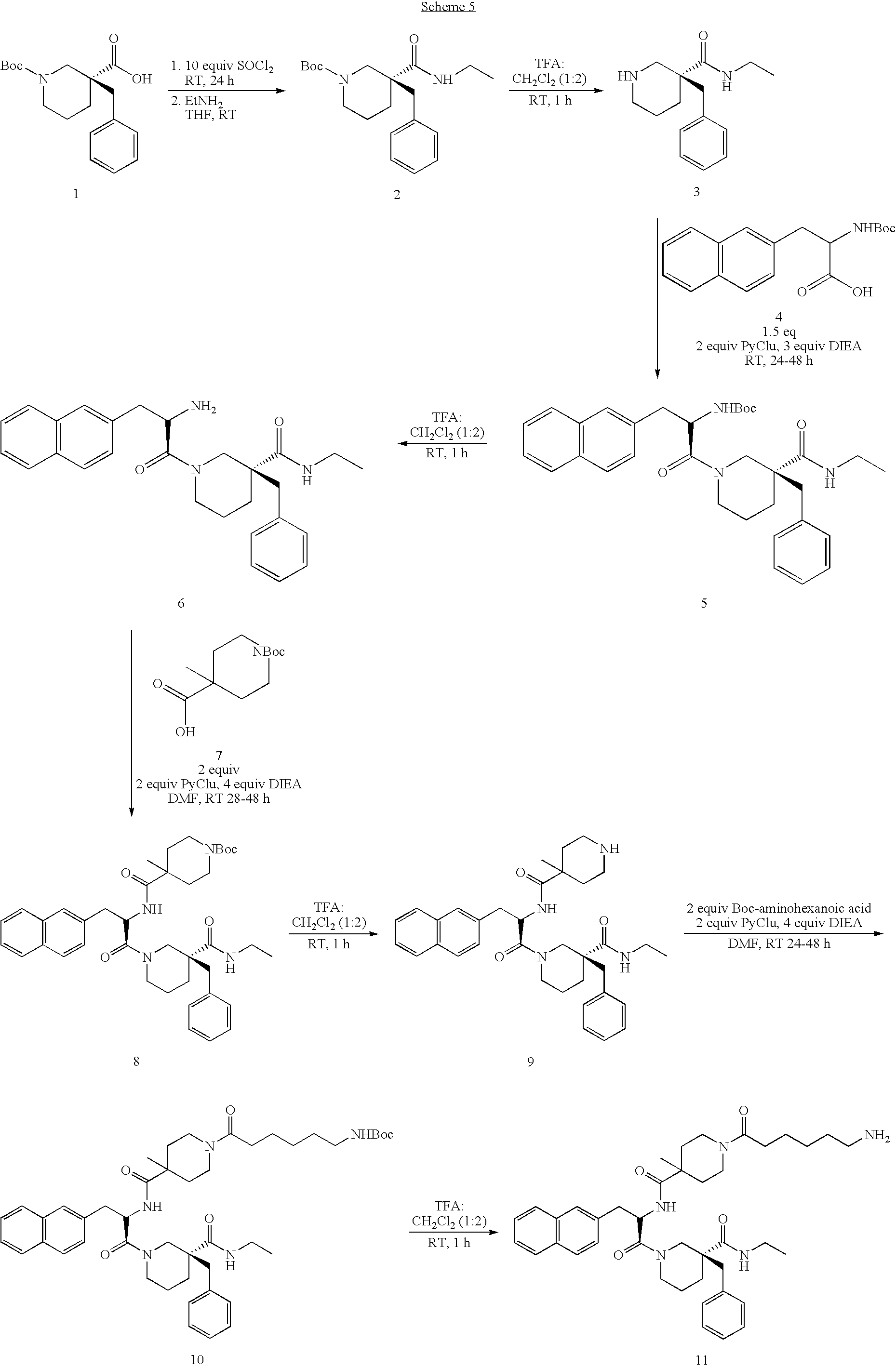

Scheme 5 describes the synthesis of intermediate 5 and compound 11.

Synthesis of Intermediate 5: 1,3-piperidine carboxylic acid, 3-(phenylmethyl)-, 1-(1,1-dimethylethylester)(3S), Compound 1 (1 g, 3.13 mmol) was dissolved in thionyl chloride (2.29 mL, 31.3 mmol), stirred overnight at room temperature, and then concentrated to dryness under reduced pressure. The crude material was directly reacted with a 2M ethylamine in THF solution (14.80 mL, 29.6 mmol) until the reaction was complete as indicated by LC-MS to give 2. The crude reaction mixture was concentrated under reduced pressure and the residue dissolved in CH₂Cl₂ (20 mL) and treated with TFA (10 mL) at room temperature for 1 hour, or until removal of the Boc protecting group was complete as monitored by LC-MS to give 3. The TFA/CH₂Cl₂ was removed under reduced pressure, and any residual TFA was neutralized by the addition of DIEA. (R)—N-Boc-2-napthylalanine, Compound 4, (0.691 g, 2.19 mmol), PyClu (0.972, 2.92 mmol), and DIEA (0.567 g, 4.38 mmol) was dissolved in DMF (1 mL), added to 3, and stirred at room temperature for 24-48 h, until completion of the coupling as indicated by LC-MS. The product mixture was concentrated under reduced pressure and the oily residue was diluted with acetonitrile/water and purified by RP-HPLC (gradient 5% B for 5 min, then 5-95 over 18 min). The collected fractions were frozen, and freeze-dried to give intermediate 5 as an off-white solid.

Synthesis of Compound 11: Compound 5 (50.2 mg, 0.092 mmol) was dissolved in CH₂Cl₂ (20 mL) and treated with TFA (10 mL) at room temperature for 1 hour, or until removal of the Boc protecting group was complete as monitored by LC-MS to give 6. The TFA/CH₂Cl₂ was removed under reduced pressure, and any residual TFA was neutralized by the addition of DIEA. 1-Boc-4-methyl-piperidine-4-carboxylic acid, Compound 7 (44.7 mg, 0.184 mmol), PyClu (61.2 mg, 0.184 mmol), and DIEA (64 μL, 0.368 mmol) was dissolved in DMF (1.0 mL), added to 6, and stirred at room temperature for 24-48 h, until completion of the coupling as indicated by LC-MS. The product mixture was concentrated under reduced pressure and the oily residue was diluted with acetonitrile/water and purified by RP-HPLC (gradient 5% B for 5 min, then 5-95 over 18 min). The collected fractions were frozen, and freeze-dried to give 8. Compound 8 (42 mg, 0.074 mmol) was dissolved in CH₂Cl₂ (20 mL) and treated with TFA (10 mL) at room temperature for 1 hour, or until removal of the Boc protecting group was complete as monitored by LC-MS to give 9. The TFA/CH₂Cl₂ was removed under reduced pressure, and any residual TFA was neutralized by the addition of DIEA. Boc-aminohexanoic acid (34.2 mg, 0.148 mmol), PyClu (49.2 mg, 0.148 mmol), and DIEA (52 μL, 0.296 mmol) was dissolved in DMF (1.0 mL), added to 9, and stirred at room temperature for 24-48 h, until completion of the coupling as indicated by LC-MS. The product mixture was concentrated under reduced pressure and the oily residue was diluted with acetonitrile/water and purified by RP-HPLC (gradient 5% B for 5 min, then 5-95 over 18 min). The collected fractions were frozen, and freeze-dried to give 10 (or Compound 1.8 in Table 2). Compound 10 was dissolved in CH₂Cl₂ (20 mL) and treated with TFA (10 mL) at room temperature for 1 hour, or until removal of the Boc protecting group was complete as monitored by LC-MS. The TFA/CH₂Cl₂ was removed under reduced pressure to give compound 11 as an oil.

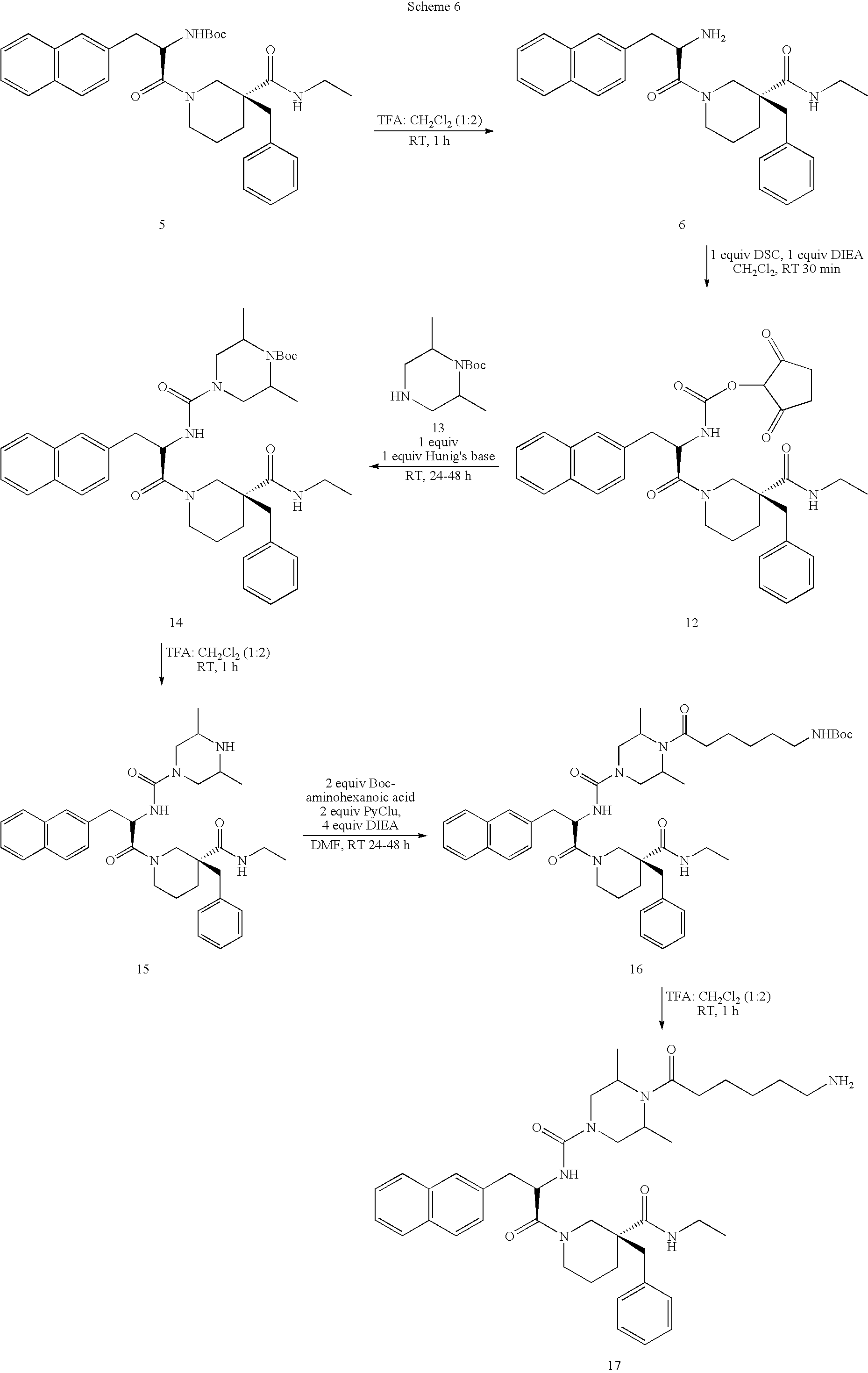

Scheme 6 describes the synthesis of Compound 17.

Compound 5 (40.6 mg, 0.075 mmol) was dissolved in CH₂Cl₂ (20 mL) and treated with TFA (10 mL) at room temperature for 1 hour, or until removal of the Boc protecting group was complete as monitored by LC-MS to give 6. The TFA/CH₂Cl₂ was removed under reduced pressure, and any residual TFA was neutralized by the addition of DIEA. N,N′-Disuccinimidyl carbonate (19.1 mg, 0.075 mmol) dissolved in CH₂Cl₂ (1.2 mL) and DIEA (13 μL, 0.075 mmol) was added to 6, and stirred at room temperature for 30 min. After the coupling was complete, as indicated by LC-MS, 1-Boc-2,6-dimethyl-piperazine 13 (15.98 mg, 0.075 mmol) and DIEA (13 μL, 0.075 mmol) was added to 12 and stirred at room temperature for 24-48 h. The product was concentrated to dryness under reduced pressure, diluted with acetonitrile/water and purified by RP-HPLC (gradient 5% B for 5 min, then 5-95 over 18 min). The collected fractions were frozen, and freeze-dried to give 14. Compound 14 was dissolved in CH₂Cl₂ (20 mL) and treated with TFA (10 mL) at room temperature for 1 hour, or until removal of the Boc protecting group was complete as monitored by LC-MS. The TFA/CH₂Cl₂ was removed under reduced pressure to give compound 15 (or Compound 1.1 in Table 2), and any residual TFA was neutralized by the addition of DIEA. Boc-aminohexanoic acid (33.3 mg, 0.144 mmol), PyClu (47.9 mg, 0.144 mmol), and DIEA (50 μL, 0.288 mmol) was dissolved in DMF (1.0 mL), added to 15, and stirred at room temperature for 24-48 h, until completion of the coupling as indicated by LC-MS. The product mixture was concentrated under reduced pressure to give an oily residue that was diluted with acetonitrile/water and purified by RP-HPLC (gradient 5% B for 5 min, then 5-95 over 18 min). The collected fractions were frozen, and freeze-dried to give 16. Compound 16 was dissolved in CH₂Cl₂ (20 mL) and treated with TFA (10 mL) at room temperature for 1 hour, or until removal of the Boc protecting group was complete as monitored by LC-MS. The TFA/CH₂Cl₂ was removed under reduced pressure, and the oily residue was diluted with acetonitrile/water and purified by RP-HPLC (gradient 5% B for 5 min, then 5-95 over 18 min). The collected fractions were frozen, and freeze-dried to give Compound 17 (or Compound 2.8 in Table 3) as a white powder.

### 6.13 Synthesis of Compounds for Conjugation

The compounds in Table 3 were synthesized. The compounds were made according to the guidance provided herein and the techniques known in the art. Preparation of compounds 2.38 (compound 30 in Scheme 9) and 2.58 (compound 21 in Scheme 7) are described as examples in Schemes 7-9). Methods to attach a linker to Compounds 2.38 (compound 32 in Scheme 9) and 2.58 (compound 23 in Scheme 7 and compound 25 in Scheme 8) are also shown.

Table 4 shows Compounds of the invention with attached linkers.

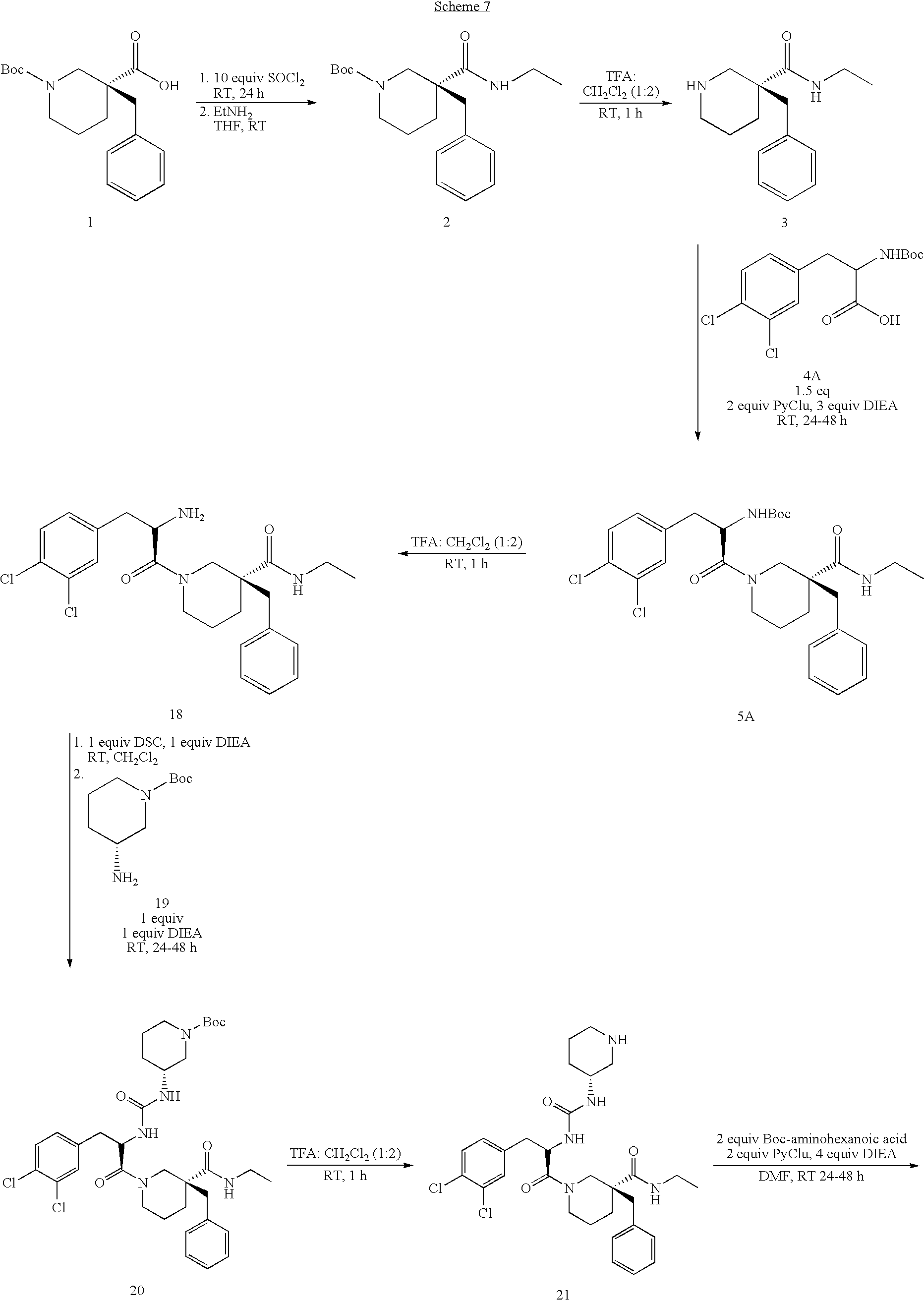

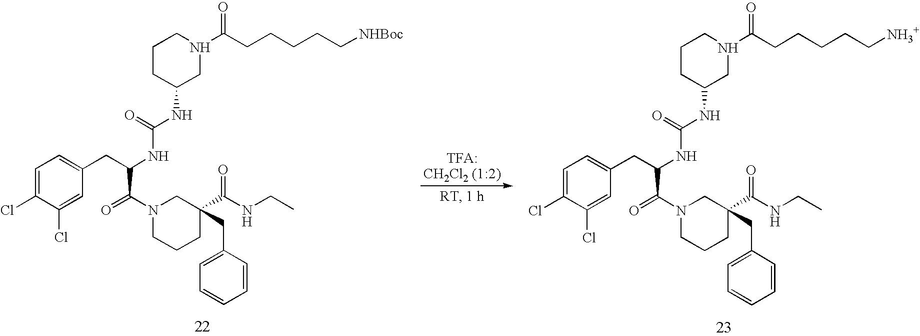

Synthesis of Intermediate 5A: 1,3-piperidine carboxylic acid, 3-(phenylmethyl)-, 1-(1,1-dimethylethylester)(3S), Compound 1 (1 g, 3.13 mmol) was dissolved in thionyl chloride (2.29 mL, 31.3 mmol), stirred overnight at room temperature, and then concentrated to dryness under reduced pressure. The crude material was directly reacted with a 2M ethylamine in THF solution (14.80 mL, 29.6 mmol) until the reaction was complete as indicated by LC-MS to give 2. The crude reaction mixture was concentrated under reduced pressure and the residue dissolved in CH₂Cl₂ (20 mL) and treated with TFA (10 mL) at room temperature for 1 hour, or until removal of the Boc protecting group was complete as monitored by LC-MS to give 3. The TFA/CH₂Cl₂ was removed under reduced pressure, and any residual TFA was neutralized by the addition of DIEA. (R)—N-Boc-3,4-dichloromethane, Compound 4A, (0.732 g, 2.19 mmol), PyClu (0.972, 2.92 mmol), and DIEA (0.567 g, 4.38 mmol) was dissolved in DMF (1 mL), added to 3, and stirred at room temperature for 24-48 h, until completion of the coupling as indicated by LC-MS. The product mixture was concentrated under reduced pressure and the oily residue was diluted with acetonitrile/water and purified by RP-HPLC (gradient 5% B for 5 min, then 5-95% B over 18 min, A=0.1% TFA/H2O and B=0.1% TFA/AcCN). The collected fractions were frozen, and freeze-dried to give intermediate 5A as an off-white solid.

Synthesis of Compound 23: Compound 18 (148 mg, 0.32 mmol) was dissolved in 1.4 mL of DCM and 1 eq of DSC (0.32 mol, 81.98 mg) was added plus 1 eq if DIEA (0.32 mmol, 55.7 uL). The reaction was monitored by LC-MS. When complete, 1 eq of (R)-1-Boc-3-amino-piperidine 19 (0.32 mmol, 64 mg) was added and 1 eq of DIEA (0.32 mol, 55.7 uL) and left to stir overnight at RT. Product formation was monitored by LC-MS. The product mixture was concentrated under reduced pressure and the oily residue was diluted with acetonitrile/water and purified by RP-HPLC (gradient 5% B for 5 min, then 5-95% B over 18 min, A=0.1% TFA/H2O and B=0.1% TFA/AcCN). The collected fractions were frozen, and freeze-dried to give 20. Compound 20 (89.8 mg, 0.13 mmol) was dissolved in CH₂Cl₂ (20 mL) and treated with TFA (10 mL) at room temperature for 1 hour, or until removal of the Boc protecting group was complete as monitored by LC-MS to give 21. The TFA/CH₂Cl₂ was removed under reduced pressure, and any residual TFA was neutralized by the addition of DIEA.

2 equiv Boc-aminohexanoic acid (70.5 mg, 0.305 mmol), 2 equiv PyClu (101.5 mg, 0.305 mmol), and 4 equiv DIEA (106 μL, 0.61 mmol) was dissolved in DMF (2.0 mL), added to 21, and stirred at room temperature for 24-48 h, until completion of the coupling as indicated by LC-MS. The product mixture was concentrated under reduced pressure and the oily residue was diluted with acetonitrile/water and purified by RP-HPLC (gradient 5% B for 5 min, then 5-95% B over 18 min, A=0.1% TFA/H2O and B=0.1% TFA/AcCN). The collected fractions were frozen, and freeze-dried to give 22. Compound 22 was dissolved in CH₂Cl₂ (20 mL) and treated with TFA (10 mL) at room temperature for 1 hour, or until removal of the Boc protecting group was complete as monitored by LC-MS. The TFA/CH₂Cl₂ was removed under reduced pressure to give compound 23 (or Compound 2.3 in Table 3) as an oil.

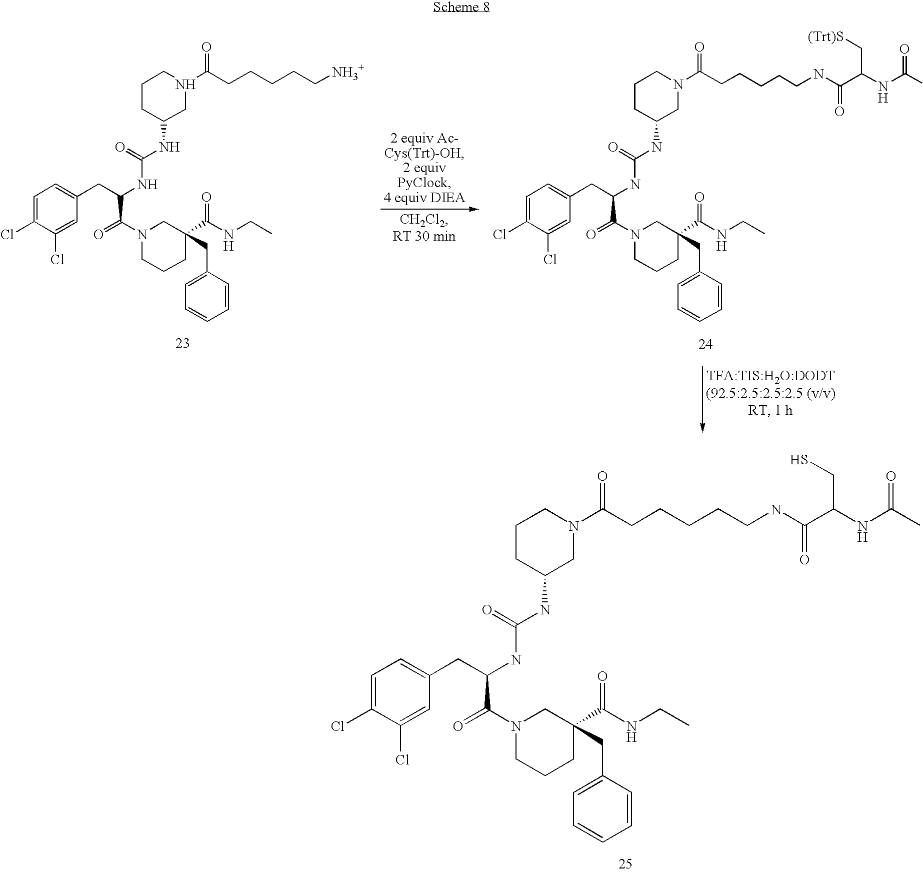

Synthesis of Compound 25: 33.63 mg of Compound 23 was dissolved in 1 mL CH₂Cl₂. 2 equiv of Ac-Cys(trt)-OH (0.096 mmol, 38.86 mg), 2 equiv PyClock (0.096 mmol, 53.3 mg), and 4 equiv of DIEA (0.19 mmol, 33.4 μL) was added. The reaction was monitored by LC-MS. The mixture was concentrated under reduced pressure. The trityl protection group on the cysteine was then removed by adding TFA/triisopropylsilane/water/DODT [92.5:2.5:2.5:2.5 (v/v)] for 1 h. The reaction was monitored by LC-MS. The mixture was concentrated under reduced pressure. The crude was dissolved in acetonitrile/water and purified by RP-HPLC (gradient 5% B for 5 min, then 5-95% B over 18 min, A=0.1% TFA/H2O and B=0.1% TFA/AcCN). The collected fractions were pooled and freeze-dried to give a white solid, Compound 25 (or Compound 2.58 in Table 3).

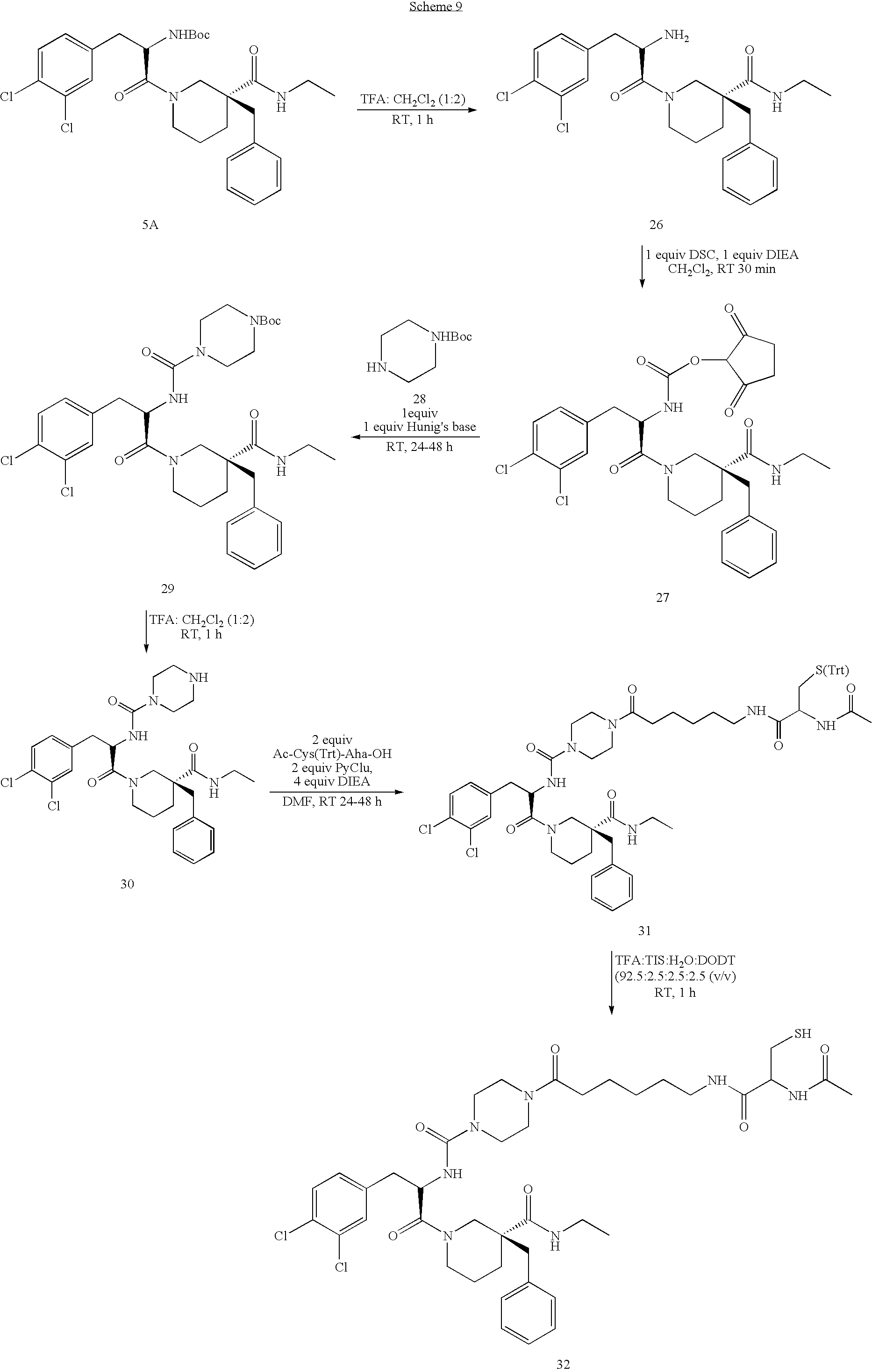

Synthesis of Compound 32: Compound 5A (180 mg, 0.32 mmol) was dissolved in CH₂Cl₂ (20 mL) and treated with TFA (10 mL) at room temperature for 1 hour, or until removal of the Boc protecting group was complete as monitored by LC-MS to give 26. The TFA/CH₂Cl₂ was removed under reduced pressure, and any residual TFA was neutralized by the addition of DIEA. N,N′-Disuccinimidyl carbonate (98 mg, 0.32 mmol) dissolved in CH₂Cl₂ (2.0 mL) and DIEA (55.7 μL, 0.32 mmol) was added to 26, and stirred at room temperature for 30 min. After the coupling was complete, as indicated by LC-MS, Boc-piperazine 28 (59.6 mg, 0.32 mmol) and DIEA (55.7 μL, 0.32 mmol) was added to 27 and stirred at room temperature for 24-48 h. The product was concentrated to dryness under reduced pressure, diluted with acetonitrile/water and purified by RP-HPLC (gradient 5% B for 5 min, then 5-95% B over 18 min, A=0.1% TFA/H2O and B=0.1% TFA/AcCN). The collected fractions were frozen, and freeze-dried to give 29. Compound 29 was dissolved in CH₂Cl₂ (20 mL) and treated with TFA (10 mL) at room temperature for 1 hour, or until removal of the Boc protecting group was complete as monitored by LC-MS. The TFA/CH₂Cl₂ was removed under reduced pressure to give 30, and any residual TFA was neutralized by the addition of DIEA. 1.5 equiv of Ac-Cys(trt)-Aha-OH (40 mg, 0.079 mmol), 2.0 equiv PyClock (35 mg, 0.104 mmol), and 4 equiv DIEA (36 μL, 0.208 mmol) was dissolved in CH₂Cl₂ (1.0 mL), added to 30, and stirred at room temperature for 30 min, or until completion of the coupling as indicated by LC-MS. The product mixture was concentrated under reduced pressure to give an oily residue. The trityl protecting group on the cysteine was then removed by adding TFA/triisopropylsilane/water/DODT [92.5:2.5:2.5:2.5 (v/v)] for 1 h. The reaction was monitored by LC-MS. The mixture was concentrated under reduced pressure. The crude was dissolved in acetonitrile/water and purified by RP-HPLC (gradient 5% B for 5 min, then 5-95% B over 18 min, A=0.1% TFA/H2O and B=0.1% TFA/AcCN). The collected fractions were pooled and freeze-dried to give a white solid, Compound 32.

### 6.14 Synthesis of Ac-Cys(Trt)-Aha-OH Linker

The Aha linker used to conjugate a compound of the invention to a carrier protein was made as a pre-formed unit according to the protocol below prior to conjugation.

0.5 g (2.75×10⁻³ mol) of Aha-OMe, was dissolved in 5 mL of CH₂Cl₂ in a 50 mL round bottom flask. 2 eq of Ac-Cys(Trt)-OH (2.2 g, 5.5×10⁻³ mol), 4 eq of DIEA (1.9 mL, 1.1×10⁻² mol), and 2 eq PyClock coupling reagent (3.057 g, 5.5×10⁻³ mol) were dissolved in 5 mL of CH₂Cl₂. After the Ac-Cys(Trt)-OH coupling mixture was dissolved, it was added to the Aha-OMe and stirred overnight. The progress of the coupling was monitored by LC/MS. The mixture was concentrated under reduced pressure. The methyl ester was removed using 1N LiOH (30 ml)/MeOH (70 ml) (3:7) for 4 h. The progress of the coupling was monitored by LC/MS. The mixture was acidified by the addition of 30 mL of 3M HCl. MeOH was then removed by rotoevaporation. 50 mL ethyl acetate and 100 mL of saturated NaCl was added, followed by extraction 4 times with 50 mL of ethyl acetate. The organic fractions were combined and the ethyl acetate was concentrated under reduced pressure. The crude was dissolved in acetonitrile/water and purified by RP-HPLC (gradient 5% B for 5 min, then 5-95 over 18 min, A=0.1% TFA/H2O and B=0.1% TFA/AcCN). The collected fractions were frozen, and freeze-dried to give a white solid.

TABLE 2
Class 2 Compounds Identified by the DBCA Screen

| Compound | |
|---|---|
| No. | Compound Structure |
| 1.1 | 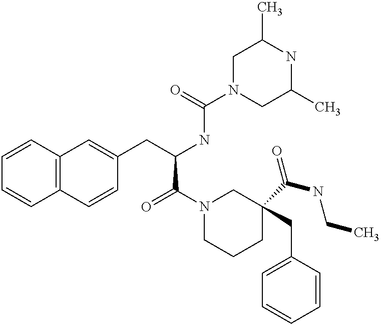 |
| 1.2 | 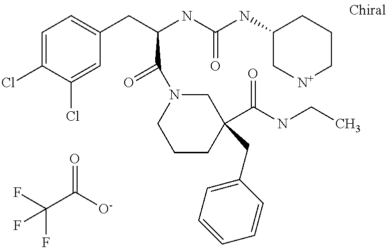 |
| 1.3 | 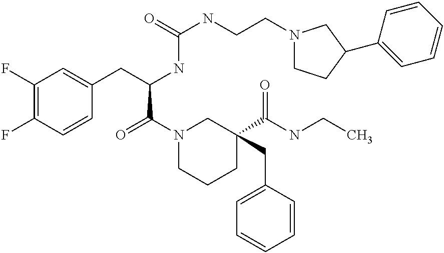 |
| 1.4 | 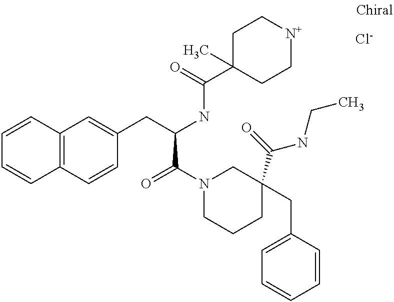 |
| 1.5 | 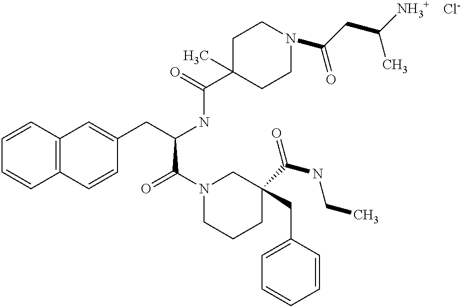 |
| 1.6 | 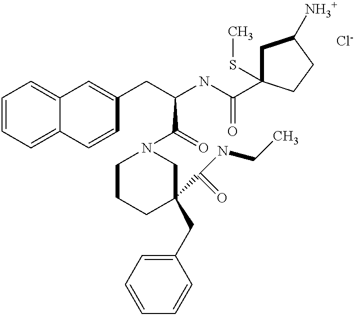 |
| 1.7 | 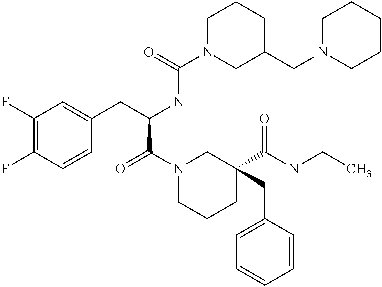 |
| 1.8 | 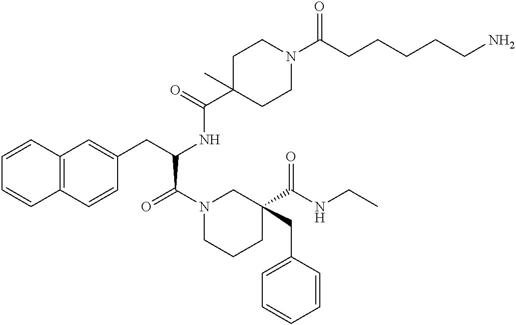 |
| 1.9 | 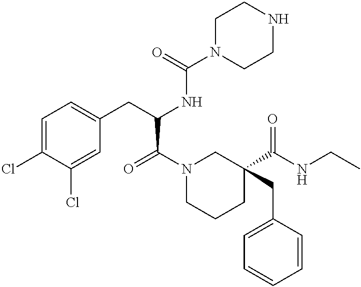 |
| 1.10 | 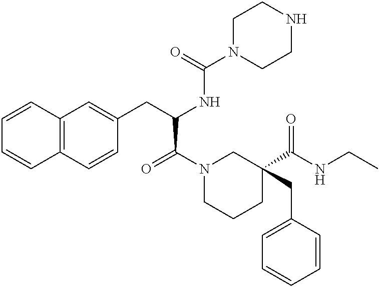 |
| 1.11 | 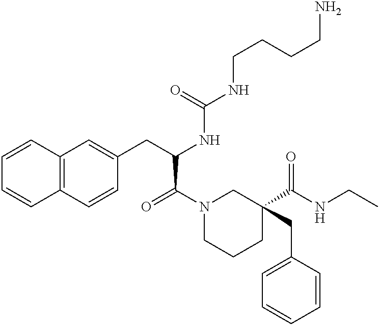 |
| 1.12 | 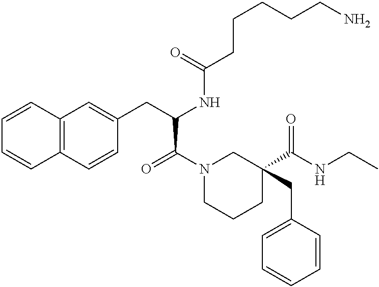 |

TABLE 3
Compounds Synthesized for Conjugation

| Number | Compound Structure |
|---|---|
| 2.1 | 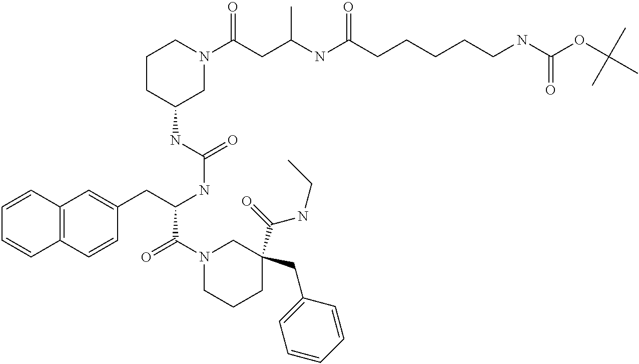 |
| 2.2 | 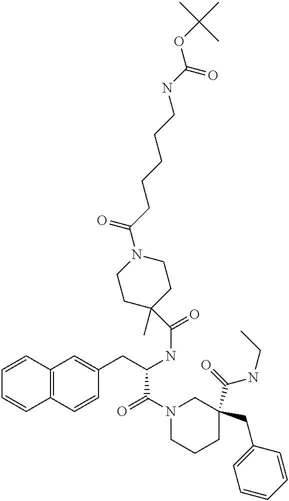 |
| 2.3 | 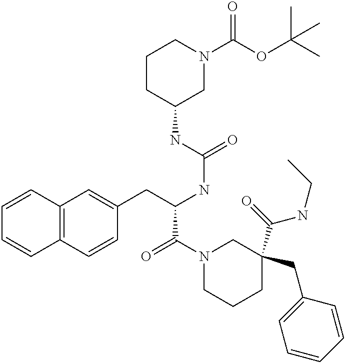 |
| 2.4 | 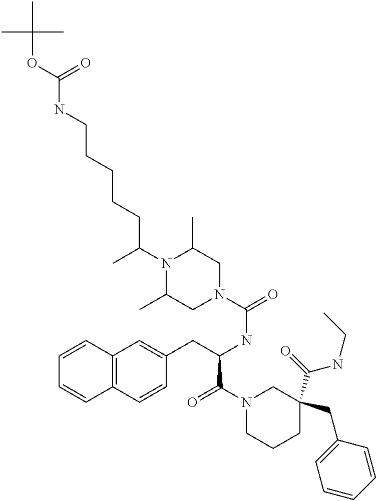 |
| 2.5 | 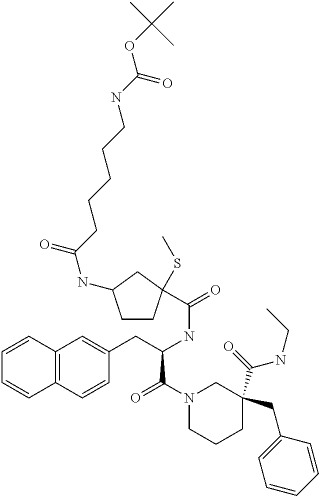 |
| 2.6 | 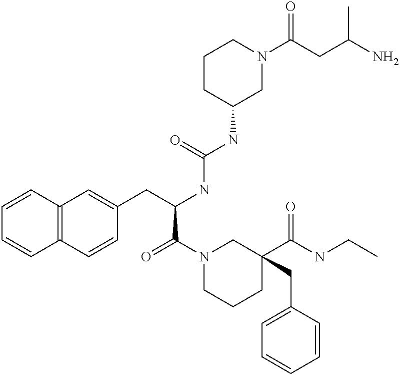 |
| 2.7 | 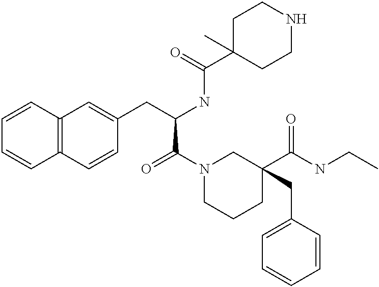 |
| 2.8 | 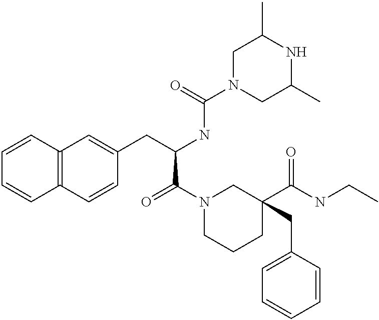 |
| 2.9 | 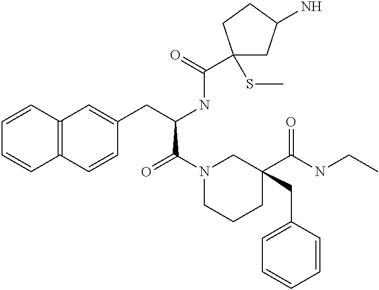 |
| 2.10 | 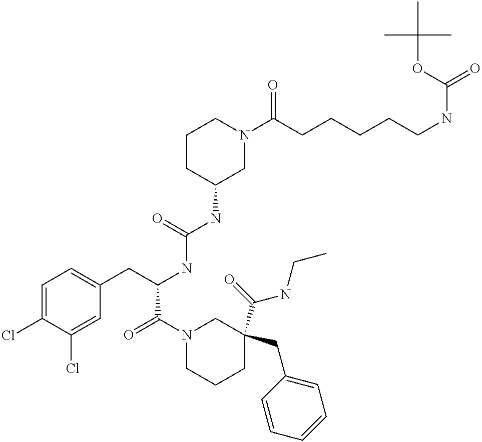 |
| 2.11 | 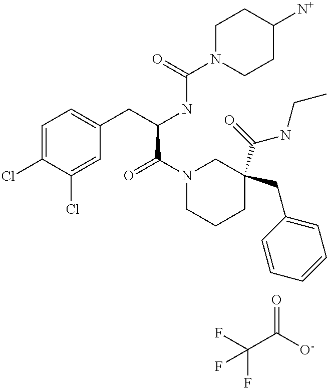 |
| 2.12 | 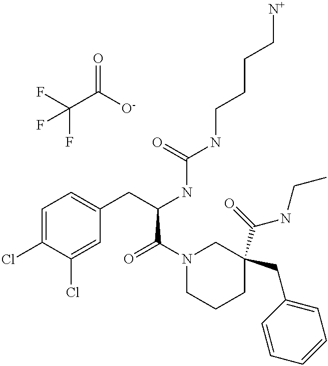 |
| 2.13 | 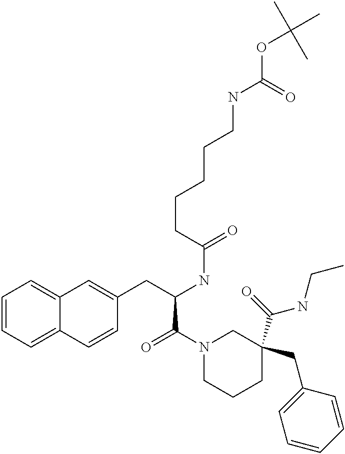 |
| 2.14 | 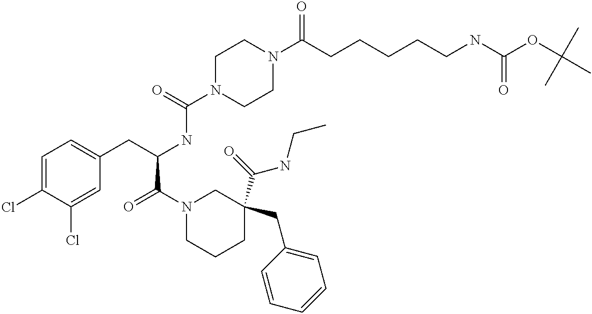 |
| 2.15 | 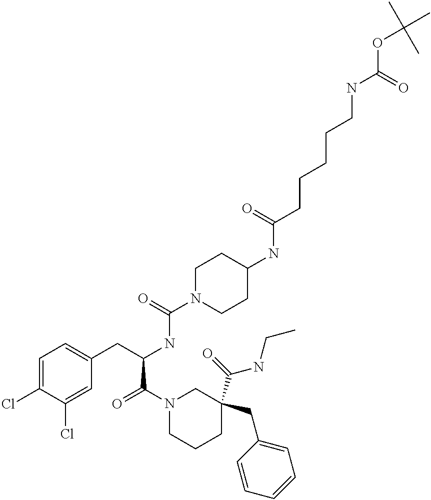 |
| 2.16 | 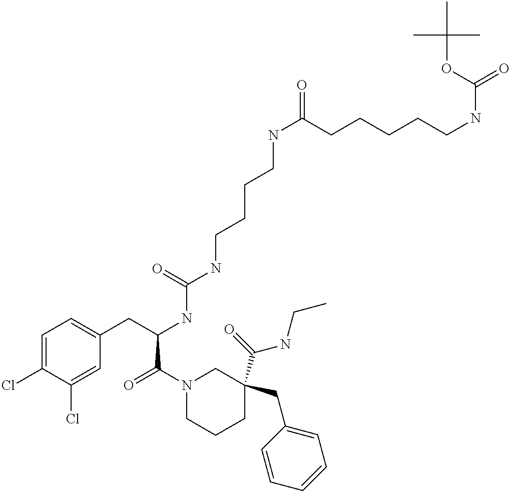 |
| 2.17 | 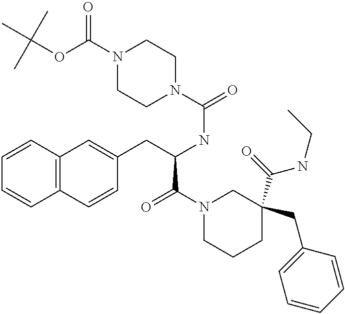 |
| 2.18 | 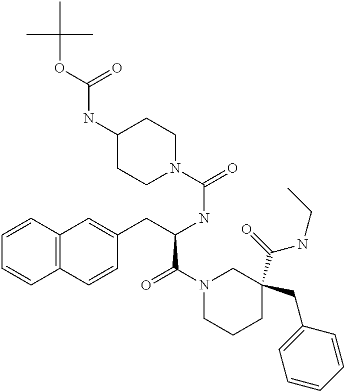 |
| 2.19 | 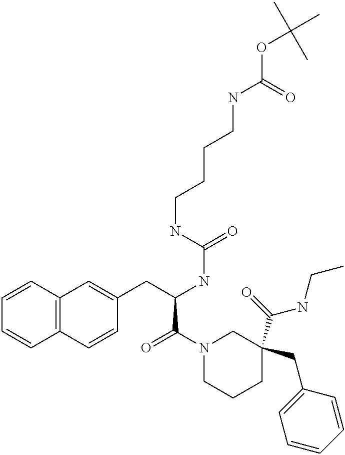 |
| 2.20 | 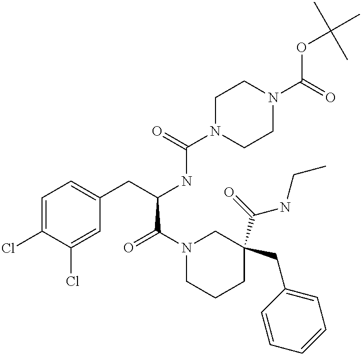 |
| 2.21 | 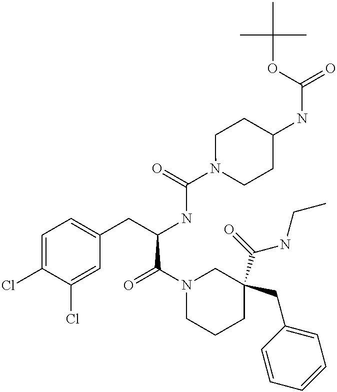 |
| 2.22 | 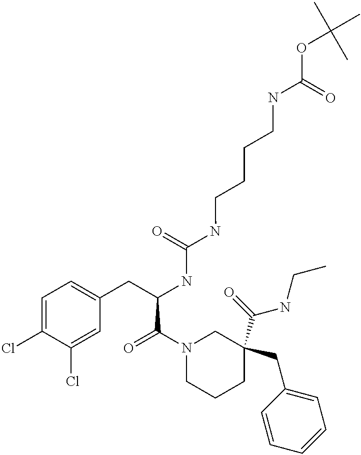 |
| 2.23 | 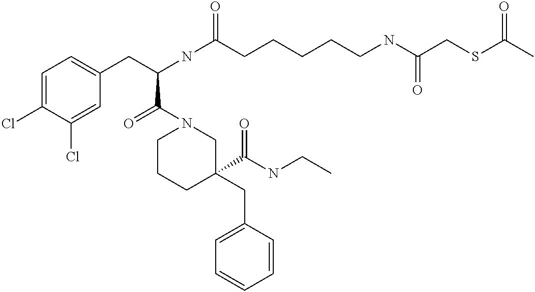 |
| 2.24 | 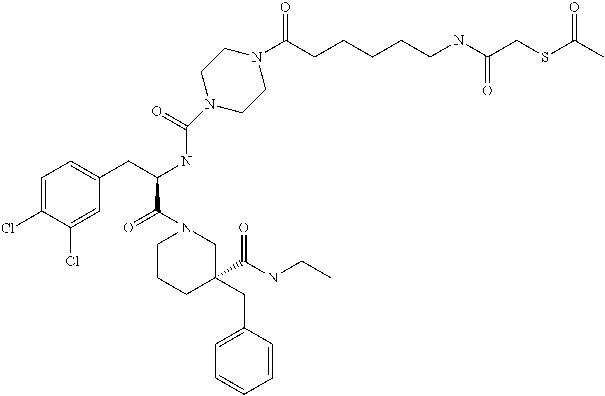 |
| 2.25 | 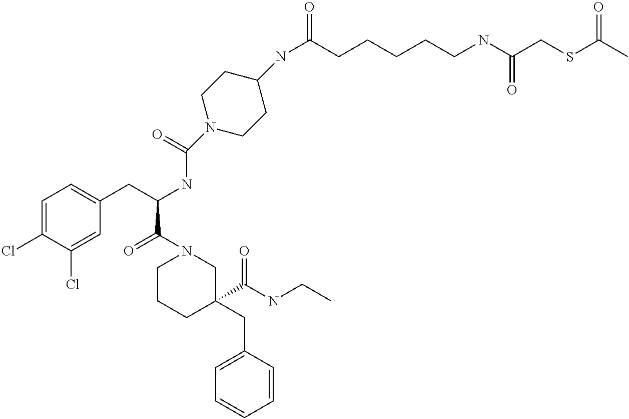 |
| 2.26 | 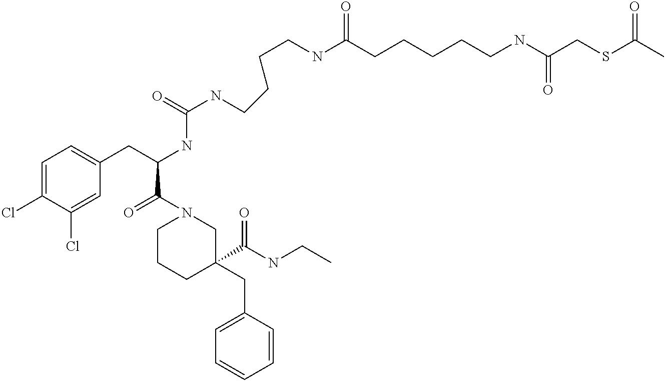 |
| 2.27 | 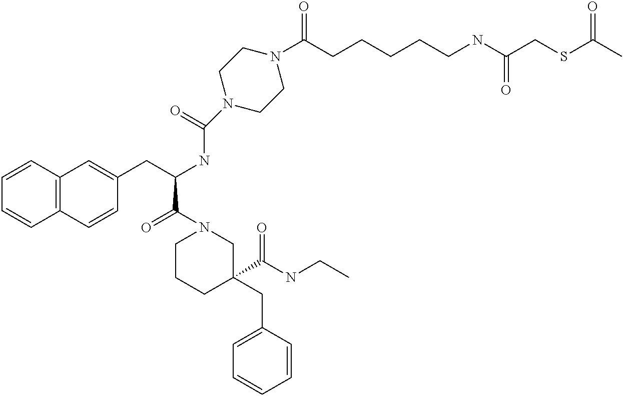 |
| 2.28 | 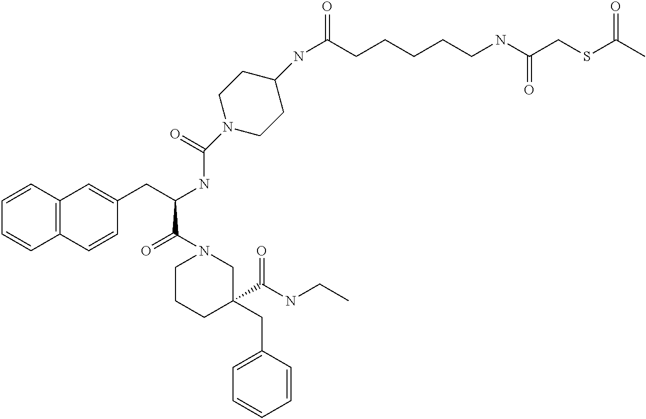 |
| 2.29 | 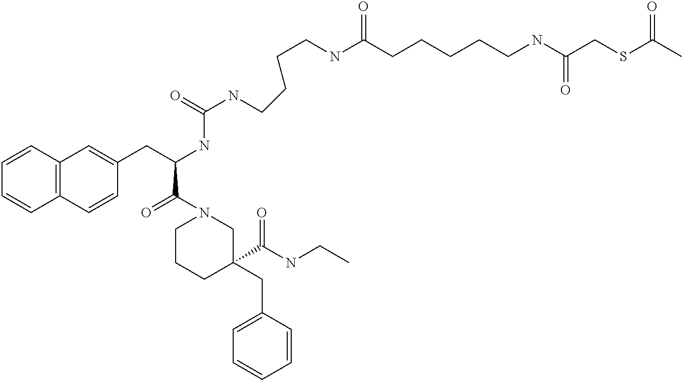 |
| 2.30 | 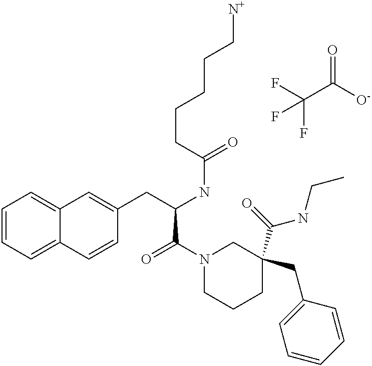 |
| 2.31 | 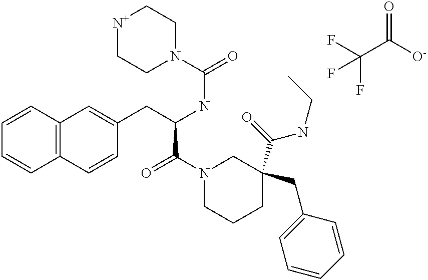 |
| 2.32 | 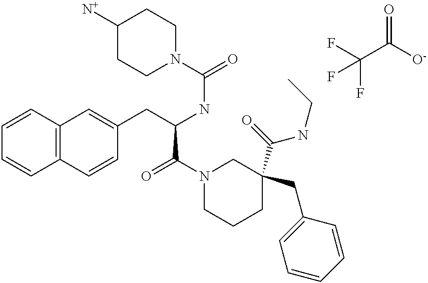 |
| 2.33 | 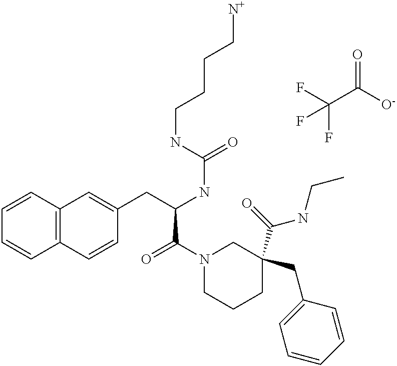 |
| 2.34 | 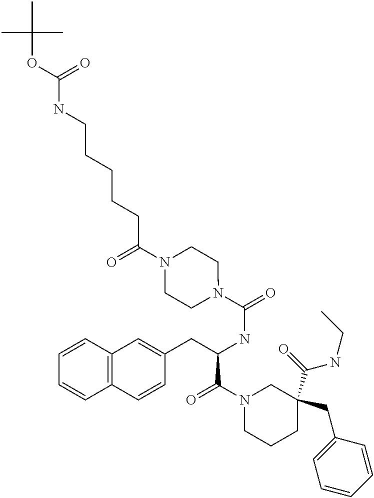 |
| 2.35 | 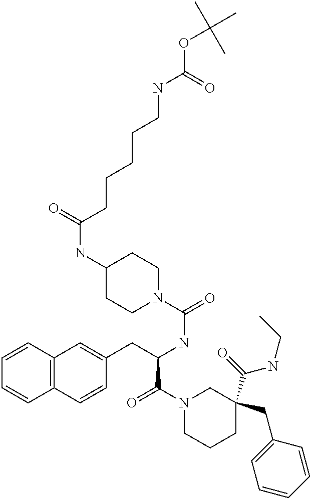 |
| 2.36 | 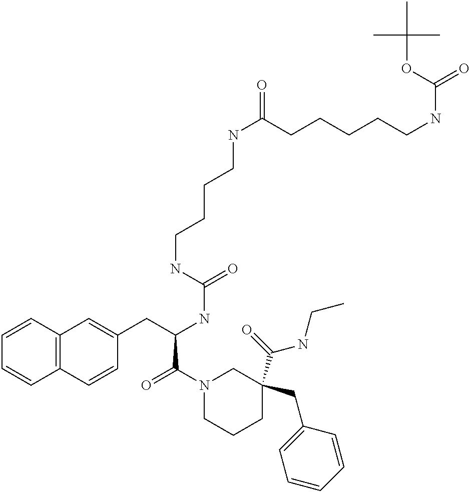 |
| 2.37 | 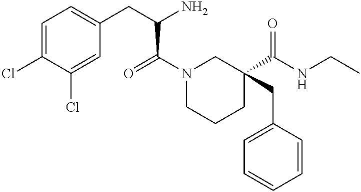 |
| 2.38 | 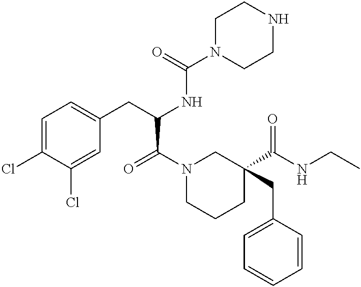 |
| 2.39 | 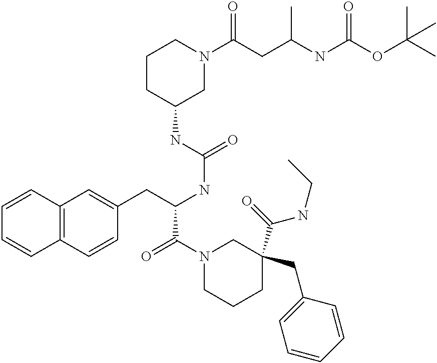 |
| 2.40 | 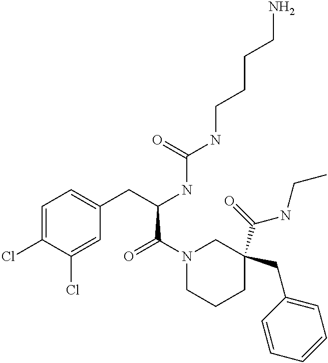 |
| 2.41 | 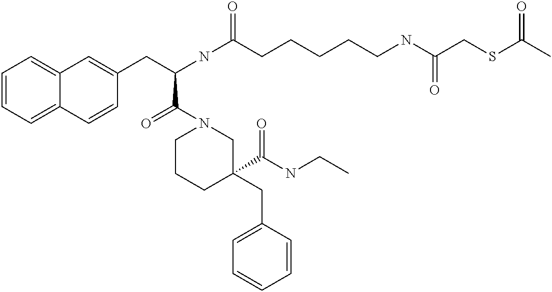 |
| 2.42 | 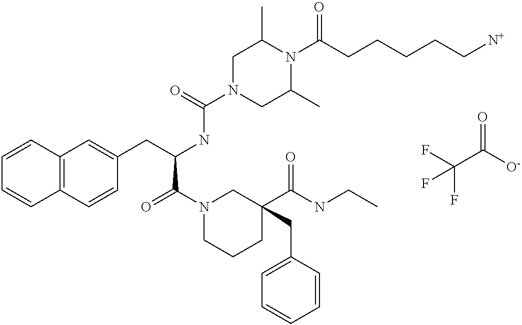 |
| 2.43 | 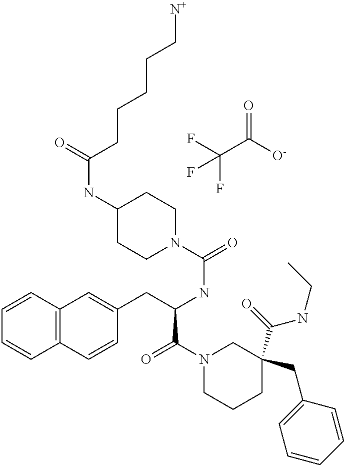 |
| 2.44 | 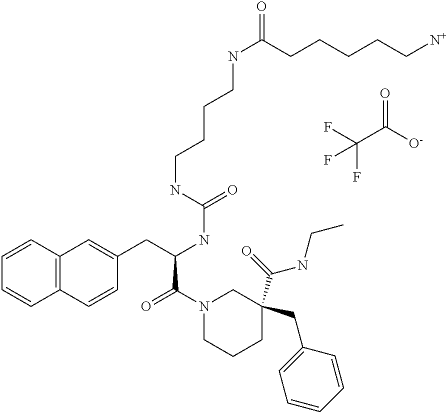 |
| 2.45 | 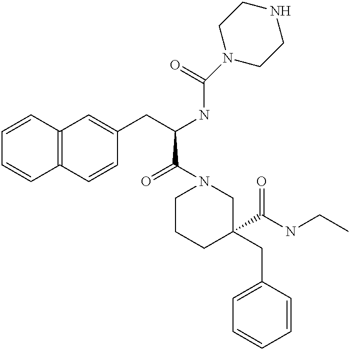 |
| 2.46 | 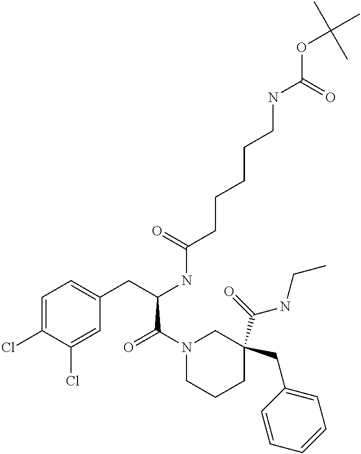 |
| 2.47 | 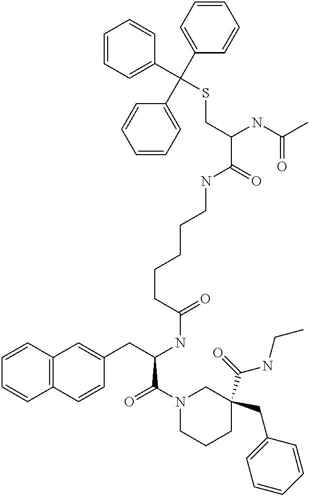 |
| 2.48 | 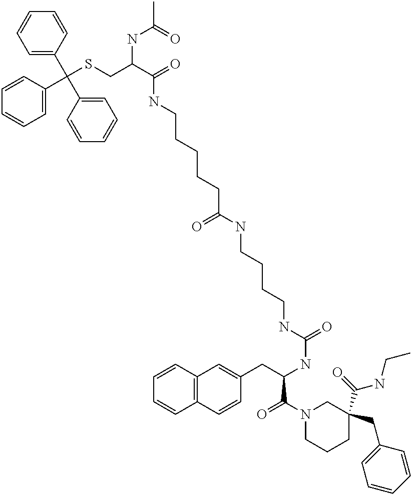 |
| 2.49 | 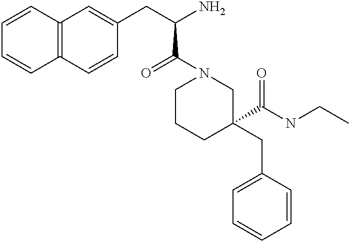 |
| 2.50 | 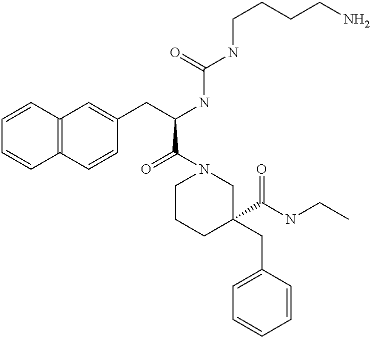 |
| 2.51 | 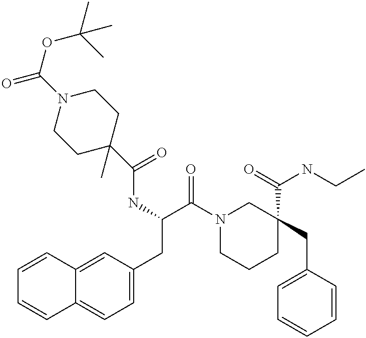 |
| 2.52 | 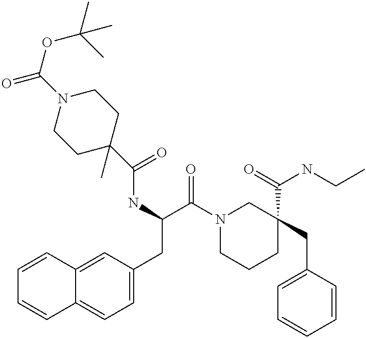 |
| 2.53 | 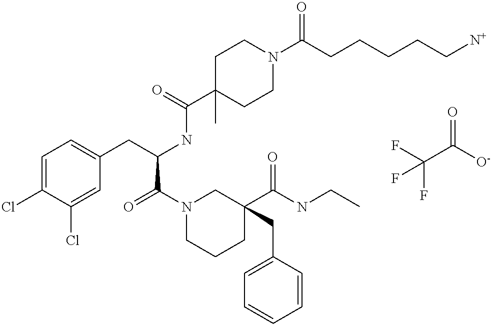 |
| 2.54 | 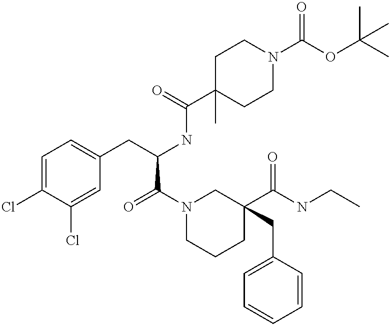 |
| 2.55 | 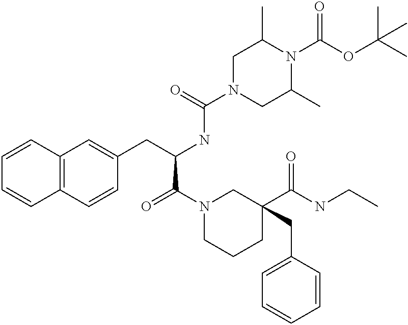 |
| 2.56 | 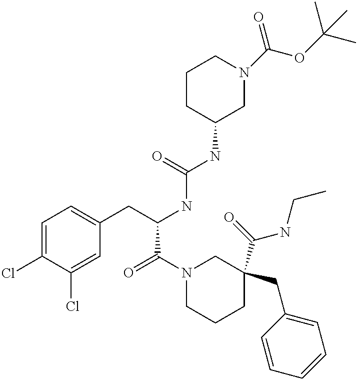 |
| 2.57 | 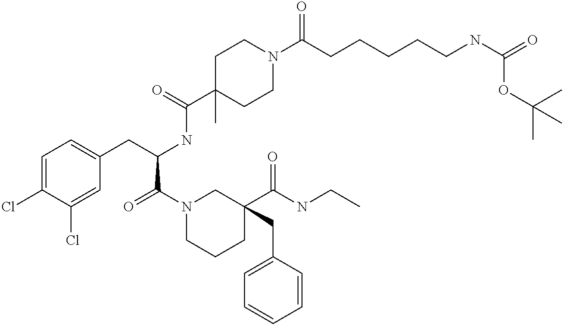 |
| 2.58 | 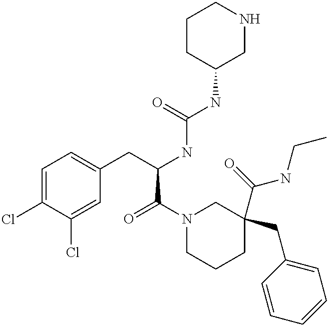 |

TABLE 4:
Compounds With Attached Linkers

| Compound | Structure with linker attached |
|---|---|
| 2.6 | 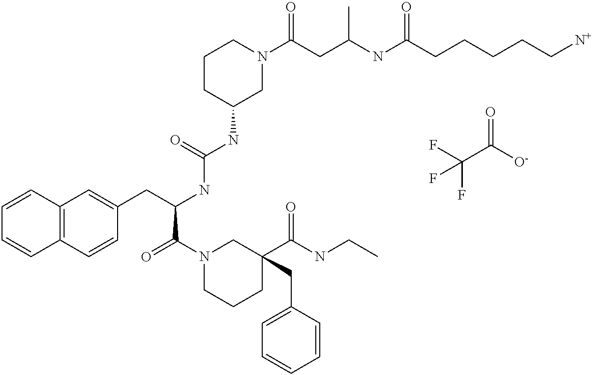 |
| 2.7 | 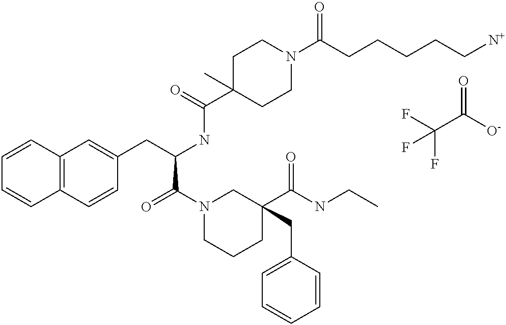 |
| 2.8 | 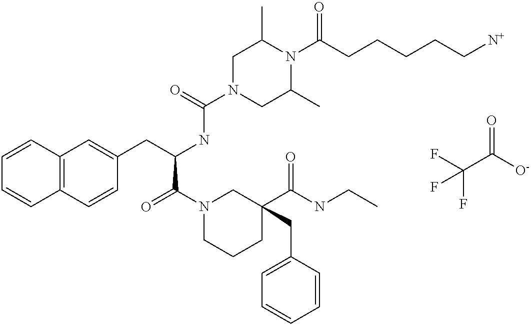 |
| 2.9 | 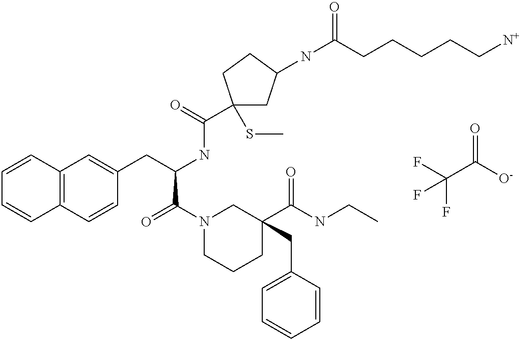 |
| 2.11 | 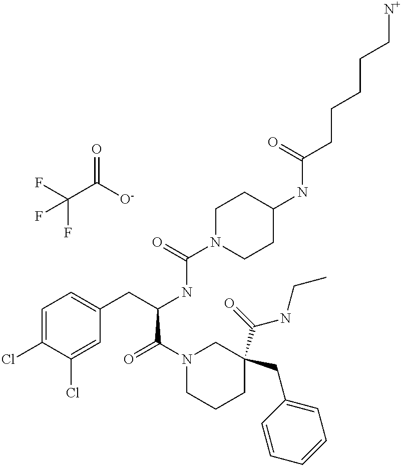 |
| 2.37 | 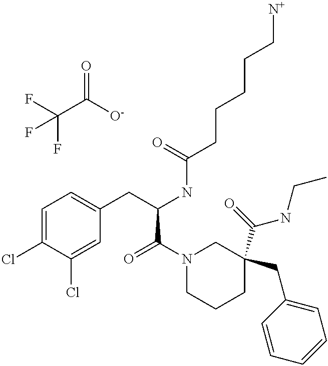 |
| 2.38 | 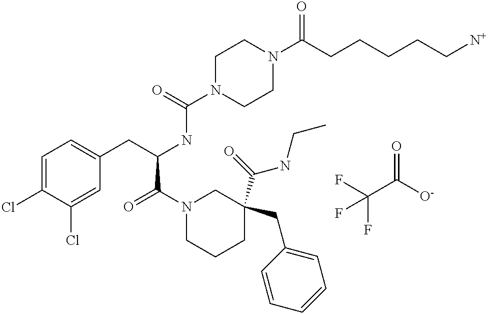 |
| 2.40 | 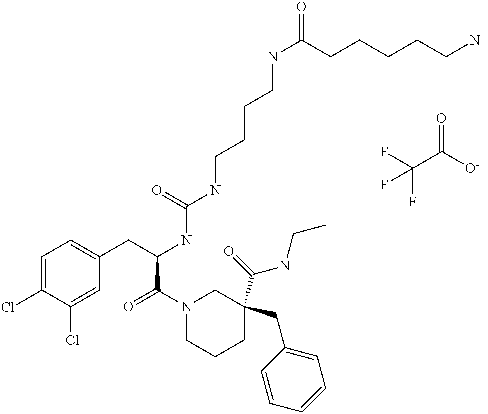 |
| 2.43 | 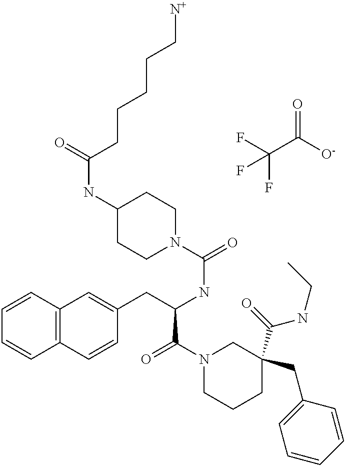 |
| 2.45 Form A | 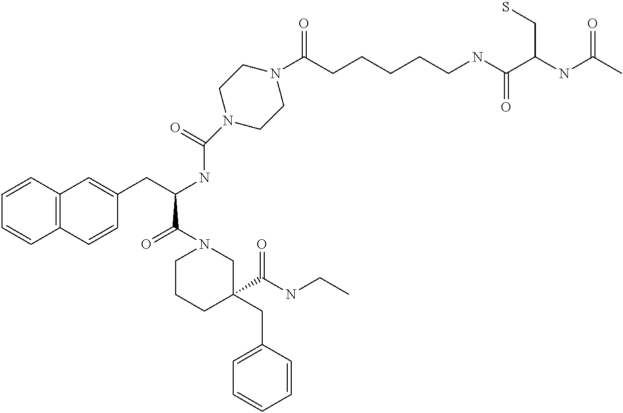 |
| 2.45 Form B | 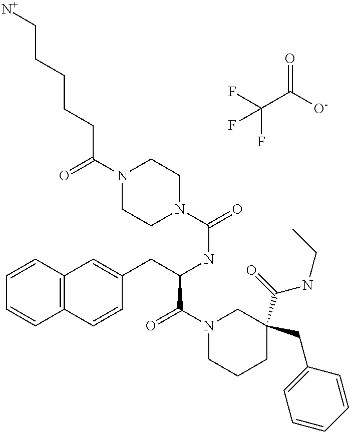 |
| 2.49 | 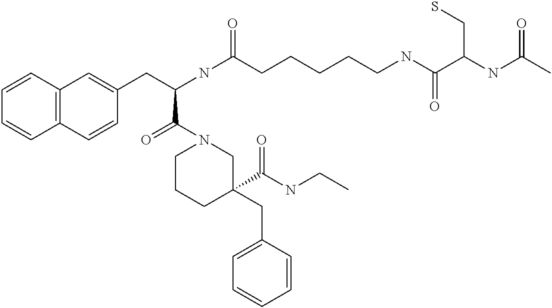 |
| 2.50 | 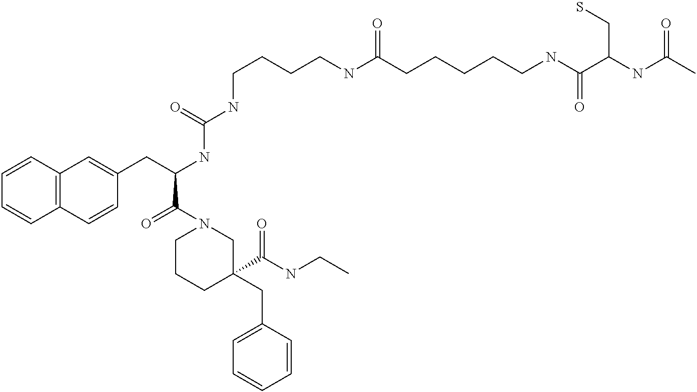 |
| 2.58 Form A | 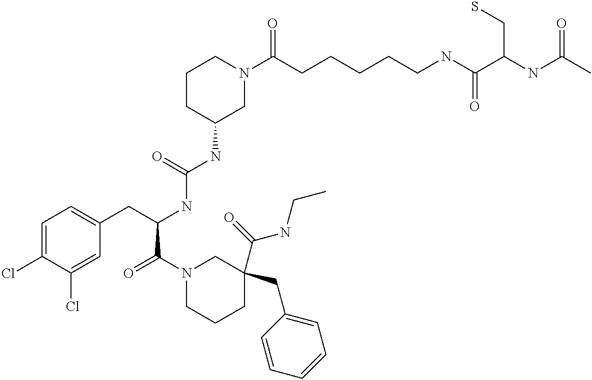 |
| 2.58 Form B | 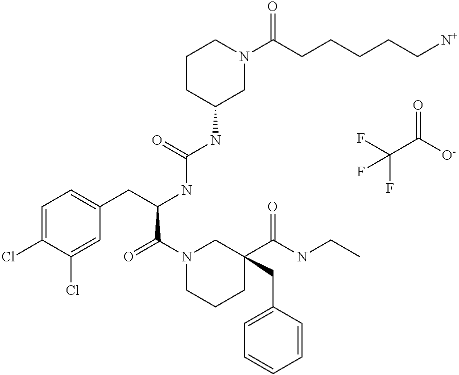 |

1. A method of identifying a small molecule mimotope of a pathogen-derived antigen comprising:
a) combining the following components
i) an antibody that binds to the antigen;
ii) the pathogen-derived antigen or a fragment or conformational mimetic thereof wherein said fragment or conformational mimetic has a conformation that is substantially similar to that of the antigen such that the antibody binds to the fragment or conformational mimetic; and
iii) a small molecule test compound;
b) incubating the components under conditions which allow components i and ii to bind;
c) measuring the amount of binding between components i and ii, wherein a decrease in binding between components i and ii in the presence of the test compound as compared to in the absence of the test compound indicates the test compound binds to component i or ii and is a candidate compound;
d) determining to which component the candidate compound binds,
wherein binding to the antibody indicates that the candidate compound is a mimotope of the pathogen-derived antigen.

2. The method of claim 1, wherein binding of the antibody to the pathogen decreases or inhibits virulence of the pathogen.

3. The method of claim 1, wherein the antigen is a fragment or conformational mimetic of the pathogen-derived antigen.

4. The method of claim 1, wherein the pathogen-derived antigen is from a pathogen selected from the group consisting of HIV, HCV, and Neisseria meningitidis.

5. The method of claim 1, wherein the pathogen-derived antigen is a polypeptide.

6. The method of claim 5, wherein the pathogen-derived antigen is a gp41 polypeptide from HIV or a 5-helix polypeptide.

7. The method of claim 1, wherein the pathogen-derived antigen is a carbohydrate.

8. (canceled)

9. The method of claim 2, wherein the antibody is a neutralizing antibody.

10. The method of claim 6, wherein the antibody is D5.

11. The method of claim 1, wherein the antibody binds to polysialic acid on Neisseria meningitides capsules but not to NCAM-associated polysialic acid.

12. A method of vaccinating an individual against a pathogen comprising administering the mimotope of the pathogen-derived antigen of claim 1 or a derivative thereof to the individual.

13. A method of vaccinating an individual against HIV comprising administering a mimotope of gp41 or a derivative thereof to the individual, wherein the mimotope is selected from the group consisting of the compounds of any of Tables 2-4.

14. The method of claim 12 wherein the mimotope is conjugated to an immunogenic carrier protein.

15. The method of claim 14, wherein the carrier protein is OMPC.

16. A method of identifying a small molecule mimotope of a self-antigen or a small molecule antagonist of an autoantibody comprising:
e) combining the following components
i) an autoantibody that binds to the self-antigen, wherein the auto antibody is associated with a pathological condition;
ii) the self-antigen or a fragment or conformational mimetic thereof that has a conformation that is substantially similar to that of the self-antigen such that the autoantibody binds to the fragment or conformational mimetic; and
iv) a small molecule test compound;
f) incubating the components under conditions which allow components i and ii to bind;
g) measuring the amount of binding between components i and ii, wherein a decrease in binding between components i and ii in the presence of the test compound as compared to in the absence of the test compound indicates the test compound binds to component i or ii and is a candidate compound;
h) determining to which component the candidate compound binds,
wherein binding to the autoantibody indicates that the candidate compound is a mimotope of the self-antigen and wherein binding to the self-antigen, fragment or conformational mimetic thereof indicates that the candidate compound is an antagonist of the autoantibody.

17. The method of claim 16, wherein the pathological condition is selected from the group consisting of myasthenia gravis, rheumatoid arthritis, lupus erythematosis, diabetes mellitus type 1, and multiple sclerosis.

18. A method of treating an autoimmune disorder comprising administering to an individual in need thereof the mimetic or the antagonist of claim 16 or a derivative thereof.

19. The method of claim 18, wherein the individual is further administered one or more drugs selected from the group consisting of corticosteroid drugs, nonsteroidal anti-inflammatory drugs, and immunosuppressant drugs.

20. A compound of Formula I 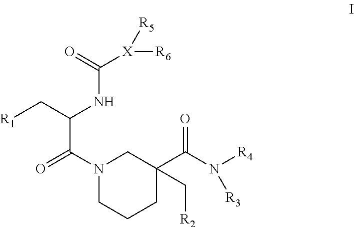
or a pharmaceutically acceptable salt thereof, wherein
R₁ is an optionally substituted aryl or heteroaryl; R₂ is an optionally substituted aryl or heteroaryl;
R₃ is either H or a C₁-C₆ alkyl;
R₄ is either H or a C₁-C₆ alkyl;
X is either N or C;
R₅ is selected from the group consisting of:
H,
(C═O)OC₁-C₆ alkyl,
(C═O)OC₁-C₆ cyloalkyl,
(C═O)OC₁-C₆ aryl,
(C═O)OC₁-C₆ heterocyclyl,
(C═O)OC₁-C₆ alkyl,
aryl,
C₂-C₆ alkenyl,
C₂-C₆ alkynyl,
heterocyclyl,
C₃-C₆cycloalkyl,
SO₂R^{a}, and
(C═O)NR^{b}₂,
said alkyl, cycloalkyl, aryl, heterocylyl, alkenyl, and alkynyl is optionally substituted with one or more substituents selected from R₇,
R₆ is selected from the group consisting of
H,
C₍₀₋₆₎alkyl optionally substituted with a heterocyclic ring or aryl,
said alkyl, heterocyclic ring and aryl is optionally substituted with R^{a}; or
R₅ and R₆ together with X form a monocyclic or bicyclic ring with 5-7 members in each ring and,
when X is C, it optionally contains a 1-4 heteroatoms selected from N, O and S, and
when X is N, it optionally contains 1 to 4 additional heteroatoms selected from N, O and S, said monocylcic or bicyclic ring optionally substituted with one or more substituents selected from R₇;
R₇ is selected from the group consisting of:
(C₁-C₆)alkyl,
(C═O)ᵣOₛ (C₁-C₆)alkyl,
Oᵣ(C₁-C₃)perfluoroalkyl,
S(O)ₘR^{a},
SR^{a},
(C₁-C₆)alkylene-S(O)ₘR^{a},
oxo,
OH,
halo,
CN,
(C₂-C₆)alkenyl,
(C₂-C₆) alkynyl,
(C₃-C₆)cycloalkyl,
aryl
(C₁-C₆)alkylene-aryl,
heterocyclyl,
(C₁-C₆)alkylene-heterocyclyl
N(R^{b})₂,
(C₁-C₆)alkylene-N(R^{b})₂,
C(O) R^{a},
CO₂ R^{a},
(C₁-C₆)alkylene-CO₂ R^{a},
C(O)H, and
CO₂H
(C₁-C₆)alkylene-CO₂H
said alkyl, alkenyl, alkynyl, alkylene, cycloalkyl, aryl, and heterocyclyl is optionally substituted with up to three substituents selected from R^{b}, OH, (C₁-C₆)alkoxy, halogen, CO₂H, CN, O(C═O) (C₁-C₆)alkyl, oxo, and N(R^{b})₂; and wherein each R is independently 0 or 1, each s is independently 0 or 1, and each m is independently 0, 1, or 2
R^{a} is selected from the group consisting of:
(C₁-C₆) alkyl,
(C₃-C₆) cycloalkyl,
aryl,
or heterocyclyl; and
R^{b} is selected from the group consisting of:
H,
(C₁-C₆) alkyl,
aryl,
heterocyclyl,
(C₃-C₆) cycloalkyl,
(C═O)O(C₁-C₆)alkyl,
(C═O)(C₁-C₆)alkyl or
S(O)₂ R^{a}.

21. The compound of Formula I or a pharmaceutically acceptable salt thereof, wherein
R₁ is an optionally substituted aryl;
R₂ is an optionally substituted phenyl;
R₃ is either H or a C₁-C₆ alkyl;
R₄ is either H or a C₁-C₆ alkyl;
X is either N or C;
R₅ is selected from the group consisting of:
H,
(C═O)OC₁-C₆ alkyl,
(C═O)OC₁-C₆ cyloalkyl,
(C═O)OC₁-C₆ aryl,
(C═O)OC₁-C₆ heterocyclyl,
(C═O)OC₁-C₆ alkyl,
aryl,
C₂-C₆ alkenyl,
C₂-C₆ alkynyl,
heterocyclyl,
C₃-C₆cycloalkyl,
SO₂R^{a}, and
(C═O)NR^{b}₂,
said alkyl, cycloalkyl, aryl, heterocylyl, alkenyl, and alkynyl is optionally substituted with one or more substituents selected from R₇,
R₆ is selected from the group consisting of
alkyl substituted with a heterocyclic ring or aryl, and
C₍₁₋₆₎alkyl substituted with a heterocyclic ring or aryl,
each of which is optionally substituted with R^{a}; or
R₅ and R₆ together with X form a monocyclic or bicyclic heterocycle with 5-7 members in each ring and,
when X is C, it contains at least one heteroatom selected from N, O and S and optionally containing 1 to 4 additional heteroatoms selected from N, O and S, and
when X is N, it optionally contains 1 to 4 additional heteroatoms selected from N, O and S, said monocylcic or bicyclic heterocycle optionally substituted with one or more substituents selected from R₇;
R₇ is selected from the group consisting of:
(C═O)ᵣOₛ (C₁-C₆)alkyl,
Oᵣ(C₁-C₃)perfluoroalkyl,
S(O)ₘ R^{a},
(C₁-C₆)alkylene-S(O)ₘ R^{a},
oxo,
OH,
halo,
CN,
(C₂-C₆)alkenyl,
(C₂-C₆) alkynyl,
(C₃-C₆)cycloalkyl,
aryl
(C₁-C₆)alkylene-aryl,
heterocyclyl,
(C₁-C₆)alkylene-heterocyclyl
N(R^{b})₂,
(C₁-C₆)alkylene-N(R^{b})₂,
C(O) R^{a},
CO₂ R^{a},
(C₁-C₆)alkylene-CO₂ R^{a},
C(O)H, and
CO₂H
(C₁-C₆)alkylene-CO₂H
said alkyl, alkenyl, alkynyl, alkylene, cycloalkyl, aryl, and heterocyclyl is optionally substituted with up to three substituents selected from R^{b}, OH, (C₁-C₆)alkoxy, halogen, CO₂H, CN, O(C═O) (C₁-C₆)alkyl, oxo, and N(R^{b})₂; and wherein each R is independently 0 or 1, each s is independently 0 or 1, and each m is independently 0, 1, or 2
R^{a} is selected from the group consisting of:
(C₁-C₆) alkyl,
(C₃-C₆) cycloalkyl,
aryl,
or heterocyclyl; and
R^{b} is selected from the group consisting of:
H,
(C₁-C₆) alkyl,
aryl,
heterocyclyl,
(C₃-C₆) cycloalkyl,
(C═O)O(C₁-C₆)alkyl,
(C═O)(C₁-C₆)alkyl or
S(O)₂ R^{a}.

22. (canceled)
